(19) United States
(12) Patent Application Publication
(10) Pub. No.: US 2022/0298225 A1
(43) Pub. Date: Sep. 22, 2022

(54) METHODS AND COMPOSITIONS FOR TREATING CANCER WITH COLLAGEN BINDING DRUG CARRIERS

(71) Applicant: The University of Chicago, Chicago, IL (US)

(72) Inventors: Jeffrey A. HUBBELL, Chicago, IL (US); Jun ISHIHARA, Chicago, IL (US); Koichi SASAKI, Chicago, IL (US)

(73) Assignee: The University of Chicago, Chicago, IL (US)

(21) Appl. No.: 17/596,118

(22) PCT Filed: Jun. 3, 2020
(86) PCT No.: PCT/US2020/070113

§ 371 Date: Dec. 3, 2021

Related U.S. Application Data
(60) Provisional application No. 62/856,468, filed on Jun. 3, 2019.

(51) Int. Cl. C07K 14/745; A61K 47/64; A61P 35/00; A61K 45/06; A61K 38/36; A61K 39/395

(52) U.S. Cl. CPC C07K 14/745; A61K 47/643; A61K 47/64; A61P 35/00; A61K 45/06; A61K 38/36; A61K 39/39558; C07K 2319/31; C07K 2319/30

(57) ABSTRACT

This disclosure relates to tumor-targeted drug carriers that lead to improved anti-tumor efficacy by efficient delivery of a cytotoxic agent to the tumor microenvironment. Aspects of the disclosure relate to a polypeptide comprising an albumin or IgG Fc domain polypeptide operatively linked to a collagen binding domain. Further aspects relate to a composition comprising a polypeptide, nucleic acid, or cell of the disclosure.

### CROSS-REFERENCE TO RELATED APPLICATIONS

This application claims the benefit of priority of U.S. Provisional Patent Application No. 62/856,468 filed Jun. 3, 2019, which is hereby incorporated by reference in its entirety.

### BACKGROUND

### II. Field of the Invention

The invention generally relates to the field of medicine. More particularly, it concerns compositions and methods involving nucleotide constructs, proteins, and drug carriers for treating cancers.

### III. Background

Serum albumin (SA) is the most abundant protein in blood. A number of compounds including small molecules, peptides, and cytokines have been fused to, conjugated to, or co-formulated with SA for improved drug delivery to disease lesions. The exceptionally long plasma half-life and/or hydrophilicity of SA contributes to improved pharmacokinetics, safety, and efficacy of the drugs.

Doxorubicin (Dox) is a small molecule anticancer drug that is approved for treating a broad spectrum of cancers by the US Food and Drug Administration (FDA). Dox internalizes within cells via passive transmembrane diffusion and interferes with DNA functions, leading to death of proliferating cells. Although Dox treatment prolongs survival of some populations of patients, anti-tumor efficacy is not dramatic partially due to acquired drug resistance. The poor therapeutic index of Dox also limits its therapeutic use. Indeed, considerable toxicity of Dox has been reported in the clinic, including bone marrow suppression, excessive inflammation, and cardiotoxicity. Dox is often used in combination with other chemotherapeutic agents. Other approaches to improve efficacy and maximum tolerated dose of Dox are liposomal formulation (Doxil) and use of a maleimide derivative of Dox with a pH-sensitive cleavable linker (aldoxorubicin), which was developed to achieve conjugation with cysteine-34 (in the human sequence) of circulating SA in situ.

Although strategies have been developed for the passive targeting of cytotoxic agents to tumors, there is a need in the art for more targeted therapies that can reduce the non-specific toxicity and increase the efficacy of the cytotoxic agent while decreasing the minimum effective dose required to achieve a therapeutic effect.

### SUMMARY OF INVENTION

Here, the inventors describe tumor-targeted drug carriers that lead to improved anti-tumor efficacy by efficient delivery of a cytotoxic agent to the tumor microenvironment. Aspects of the disclosure relate to a polypeptide comprising an albumin polypeptide or IgG Fc domain polypeptide operatively linked to a collagen binding domain. Further aspects relate to a composition comprising a polypeptide, nucleic acid, or cell of the disclosure. Further aspects relate to a nucleic acid encoding for a polypeptide of the disclosure. Yet further aspects relate to a cell comprising a nucleic acid or polypeptide of the disclosure.

Further aspects of the disclosure relate to a method for making a polypeptide comprising expressing a nucleic acid of the disclosure in a cell and isolated the expressed polypeptide.

Further aspects relate to a method for treating cancer comprising administering a polypeptide, nucleic acid, or composition of the disclosure. Further aspects relate to a method for reducing non-specific toxicity of a treatment comprising a cytotoxic agent in a subject, the method comprising administering the polypeptide or composition of the disclosure to the subject. The term “non-specific toxicity” refers to toxicity or cell death of non-cancerous cells.

Further aspects relate to a method for increasing the accumulation of a cytotoxic agent in a tumor in a subject, the method comprising administering a polypeptide, nucleic acid, or composition of the disclosure to the subject.

Further aspects relate to a method for targeted delivery of a cytotoxic agent to the tumor vasculature, the method comprising administering a polypeptide or composition of the disclosure to the subject. Yet further aspects relate to a method of treating a tumor or a method of treating a tumor in a subject, the method comprising administering a polypeptide or composition of the disclosure to the tumor or subject. In some aspects, the method is for inhibiting tumor growth or tumor progression. The inhibition may be at least, at most, or about 10, 20, 30, 40, 50, 60, 70, 80, 90, 95, 99, or 100% (or any derivable range therein).

In some embodiments, the polypeptide is operatively linked to a cytotoxic agent. The term “operatively linked” refers to a covalent or non-covalent attachment. In some embodiments, the attachment is covalent. In some embodiments, the attachment is non-covalent. In some embodiments, the polypeptide is covalently linked to the cytotoxic agent. In some embodiments, the peptide is non-covalently linked to the cytotoxic agent. In some embodiments, the polypeptide is linked to the cytotoxic agent through a cleavable linker. In some embodiments, the cleavable linker comprises a pH-cleavable linker. In some embodiments, the linker comprises a hydrazone linker. In some embodiments, the linker is cleaved at a pH of less than 7.4. In some embodiments, the linker is cleaved at an acidic pH. In some embodiments, optimal cleavage of the linker is at a pH of 4.5, 5, 5.5, 6, or 6.5 (or any range therein). Optimal cleavage refers to the pH in which at least 75, 80, 85, 90, 95, or 99% of cleavage occurs in solution or in vitro at a time period of less than 6, 5, 4, 3, 2, 1, 0.5, or 0.25 hours (or any derivable range therein). In some embodiments, the polypeptide is linked to the cytotoxic agent and/or the collagen binding polypeptide through a bifunctional linker.

In some embodiments, the cytotoxic agent comprises doxorubicin. In some embodiments, the cytotoxic agent comprises a derivative of doxorubicin. In some embodiments, the cytotoxic agent comprises aldoxorubicin. In some embodiments, the cytotoxic agent is a cytotoxic agent described herein. In some embodiments, the cytotoxic agent is conjugated to the polypeptide prior to administration of the polypeptide. In some embodiments, in situ conjugation of the cytotoxic agent is excluded.

In some embodiments, the polypeptide is covalently linked to the collagen binding domain through a peptide bond. In some embodiments, the polypeptide comprises a collagen binding domain from decorin or von Willebrand factor (vWF). In some embodiments, the collagen binding domain comprises a polypeptide with at least 80% identity to SEQ ID NO:1 or a fragment thereof. In some embodiments, the collagen binding domain comprises a polypeptide with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identity to SEQ ID NO:1-4 or 11-14, or a fragment thereof. In some embodiments, the collagen binding domain comprises a polypeptide with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identity to SEQ ID NO:1, or a fragment thereof. In some embodiments, the collagen binding domain comprises a polypeptide with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identity to SEQ ID NO:2, or a fragment thereof. In some embodiments, the collagen binding domain comprises a polypeptide with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identity to SEQ ID NO:3, or a fragment thereof. In some embodiments, the collagen binding domain comprises a polypeptide with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identity to SEQ ID NO:4, or a fragment thereof. In some embodiments, the collagen binding domain comprises a polypeptide with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identity to SEQ ID NO:11, or a fragment thereof. In some embodiments, the collagen binding domain comprises a polypeptide with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identity to SEQ ID NO:12, or a fragment thereof. In some embodiments, the collagen binding domain comprises a polypeptide with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity to SEQ ID NO:13, or a fragment thereof In some embodiments, the collagen binding domain comprises a polypeptide with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity to SEQ ID NO:14, or a fragment thereof.

In some embodiments, the polypeptide is covalently linked to an albumin polypeptide. In some embodiments, the albumin polypeptide comprises a polypeptide with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to one of SEQ ID NOS:7-10.

In some embodiments, the polypeptide is covalently linked to an IgG Fc domain polypeptide. In some embodiments, the IgG Fc domain polypeptide comprises a polypeptide with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to one of SEQ ID NOS:15-18.

In some embodiments, the collagen binding domain is at the amino end of the albumin polypeptide. In some embodiments, the collagen binding domain is at the carboxy end of the albumin polypeptide. The phrase “at the amino end” or “at the carboxy end” refers to the relative position of one polypeptide to another. For example, when one polypeptide is “at the amino end” it is linked to the N-terminal amine group of the other polypeptide. However, there may be intervening sequences between the two polypeptides or domains. Similarly, a polypeptide “at the carboxy end” refers to a polypeptide linked to the carboxy terminus of another polypeptide or domain. In some embodiments, the cytotoxic agent is linked to the amino terminus of the collagen binding domain. In some embodiments, the cytotoxic agent is linked to the carboxy terminus of the collagen binding domain. In some embodiments, the cytotoxic agent is linked to the amino terminus of the albumin polypeptide. In some embodiments, the cytotoxic agent is linked to the carboxy terminus of the albumin polypeptide. In some embodiments, the collagen binding domain is at the amino end of the IgG Fc domain polypeptide.

In some embodiments, the collagen binding domain is at the carboxy end of the IgG Fc domain polypeptide. In some embodiments, the cytotoxic agent is linked to the amino terminus of the collagen binding domain. In some embodiments, the cytotoxic agent is linked to the carboxy terminus of the collagen binding domain. In some embodiments, the cytotoxic agent is linked to the amino terminus of the IgG Fc domain polypeptide. In some embodiments, the cytotoxic agent is linked to the carboxy terminus of the IgG Fc domain polypeptide.

In some embodiments, the polypeptide comprises a linker between the IgG Fc domain polypeptide and the collagen binding domain. In some embodiments, the linker comprises glycine and serine amino acid residues. In some embodiments, the linker comprises GGGS, (GGGS)ₙ, or (GGGS)₂.

In some embodiments, the polypeptide comprises a linker between the albumin polypeptide and the collagen binding domain. In some embodiments, the linker comprises glycine and serine amino acid residues. In some embodiments, the linker comprises GGGS, (GGGS)ₙ, or (GGGS)₂. In some embodiments, n is 2, 3, 4, 5, 6, 7, 8, 9, 10, 11, 12 or more (or any range derivable therein).

In some embodiments, the polypeptide is not operatively linked to a particle, nanovesicle, or liposome. In some embodiments, the polypeptide is not operatively linked to a nanoparticle or a solid support, such as a microplate or bead. In some embodiments, the composition does not comprise a liposome, particle, or nanovescicle. In some embodiments, the composition does not comprise a nanoparticle or a solid support, such as a microplate or a bead.

In some embodiments, the polypeptide comprises at least two collagen binding domains. In some embodiments, the polypeptide comprises at least 2, 3, 4, 5, 6, 7, or 8 collagen binding domains. The collagen binding domains may be in tandem or at both the amino and carboxy terminus of the albumin polypeptide or IgG Fc domain polypeptide.

In some embodiments, the ratio of cytotoxic agent to albumin is 3:1. In some embodiments, the ratio of cytotoxic agent to albumin is at least, at most, or exactly 0.5:1, 1:1, 1.5:1, 2:1, 2.5:1, 3:1, 3.5:1, 4:1, 4.5:1, 5:1, 5.5:1, 6:1, 7:1, 8:1, 9:1, or 10:1 (or any derivable range therein). In some embodiments, the ratio of albumin polypeptide to collagen binding domain is 1:1, 1:2, 1:3, 1:4, 4:1, 3:1, or 2:1 (or any range derivable therein).

In some embodiments, the ratio of cytotoxic agent to IgG Fc domain is 3:1. In some embodiments, the ratio of cytotoxic agent to IgG Fc domain is at least, at most, or exactly 0.5:1, 1:1, 1.5:1, 2:1, 2.5:1, 3:1, 3.5:1, 4:1, 4.5:1, 5:1, 5.5:1, 6:1, 7:1, 8:1, 9:1, or 10:1 (or any derivable range therein). In some embodiments, the ratio of IgG Fc domain polypeptide to collagen binding domain is 1:1, 1:2, 1:3, 1:4, 4:1, 3:1, or 2:1 (or any range derivable therein).

In some embodiments, the subject has cancer. In some embodiments, the subject has breast cancer or colon cancer or the tumor is a breast or colon tumor. In some embodiments, the subject has a cancer recited herein or a tumor from a cancer recited herein. In some embodiments, the cancer or tumor comprises a solid tumor. In some embodiments, hematological tumors or cancers are excluded.

In some embodiments, the non-specific toxicity is reduced compared to the toxicity of the same cytotoxic agent linked to albumin and unlinked to collagen binding domain. For example, the non-specific toxicity may be reduced by at least 10, 20, 30, 40, 50, 60, 70, 80, 90, or 95% in the polypeptide comprising the collagen binding domain compared to the same polypeptide without the collagen binding domain. In some embodiments, the accumulation of the cytotoxic agent in the tumor is increased compared to the dose of the same cytotoxic agent linked to albumin and unlinked to collagen binding domain. In some embodiments, the increase is at least 10, 20, 30, 40, 50, 60, 70, 80, 90, or 95%.

In some embodiments, the non-specific toxicity is reduced compared to the toxicity of the same cytotoxic agent linked to IgG Fc domain and unlinked to collagen binding domain. For example, the non-specific toxicity may be reduced by at least 10, 20, 30, 40, 50, 60, 70, 80, 90, or 95% in the polypeptide comprising the collagen binding domain compared to the same polypeptide without the collagen binding domain. In some embodiments, the accumulation of the cytotoxic agent in the tumor is increased compared to the dose of the same cytotoxic agent linked to IgG Fc domain and unlinked to collagen binding domain. In some embodiments, the increase is at least 10, 20, 30, 40, 50, 60, 70, 80, 90, or 95%.

In some embodiments, the method further comprises administration of an additional cancer therapy. In some embodiments, the subject has or will receive an immunotherapy. In some embodiments, the subject has been determined to be non-responsive to the immunotherapy. In some embodiments, the subject has refractory cancer. In some embodiments, the subject is one that experienced toxicity associated with the previous therapy or previous immunotherapy. In some embodiments, the method further comprises administration of an immunotherapy. In some embodiments, the immunotherapy is administered before, after, or concurrent with the polypeptide. In some embodiments, the immunotherapy comprises checkpoint inhibitor therapy. In some embodiments, the checkpoint inhibitor therapy comprises mono checkpoint inhibitor therapy, which indicates that only one checkpoint inhibitor is administered. In some embodiments, the checkpoint inhibitor therapy comprises combination checkpoint inhibitor therapy, which indicates that at least two checkpoint inhibitors, such as an inhibitor to PD-1 and an inhibitor to CTLA-4 is administered. In some embodiments, the checkpoint inhibitor therapy comprises a PD-1 antibody. In some embodiments, the checkpoint inhibitor therapy comprises one or more checkpoint inhibitors described herein.

In some embodiments, the polypeptide and additional therapy is administered in the same composition. In some embodiments, the polypeptide and additional therapy are administered in separate compositions. In some embodiments, compositions of the disclosure further comprise one or more immune checkpoint inhibitors. In some embodiments, compositions of the disclosure comprise a PD-1 antibody. In some embodiments, compositions of the disclosure comprise a CTLA-4 antibody. In some embodiments, compositions of the disclosure comprise a PD-1 and CTLA-4 antibody.

In some embodiments, the polypeptide or composition is administered systemically. In some embodiments, the polypeptide or composition is administered by intravenous injection. In some embodiments, the polypeptide or composition is administered intratumorally or peritumorally. In some embodiments, the polypeptide or composition is administered through a route of administration described herein.

In some embodiments, the administered dose of the cytotoxic agent is less than the minimum effective dose of the cytotoxic agent unconjugated to collagen binding domain. In some embodiments, the administered dose of the cytotoxic agent is at least 10, 20, 30, 40, 50, 60, 70, 80, or 90% (or any derivable range therein) less than the minimum effective dose of the cytotoxic agent unconjugated to collagen binding domain. In some embodiments, the administered dose of the cytotoxic agent is less than the minimum effective dose of the cytotoxic agent conjugated to an albumin polypetide and unconjugated to collagen binding domain. In some embodiments, the administered dose of the cytotoxic agent is at least 10, 20, 30, 40, 50, 60, 70, 80, or 90% (or any derivable range therein) less than the minimum effective dose of the cytotoxic agent conjugated to an albumin polypetide and unconjugated to collagen binding domain. In some embodiments, the administered dose of the cytotoxic agent is less than the minimum effective dose of the cytotoxic agent conjugated to an IgG Fc domain polypetide and unconjutated to collagen binding domain. In some embodiments, the administered dose of the cytotoxic agent is at least 10, 20, 30, 40, 50, 60, 70, 80, or 90% (or any derivable range therein) less than the minimum effective dose of the cytotoxic agent conjugated to an IgG Fc domain polypetide and unconjutated to collagen binding domain. In some embodiments, the subject has been previously treated with a cytotoxic agent. In some embodiments, the subject has been determined to be non-responsive to the previous treatment or wherein the subject experienced non-specific toxicity to the previous treatment. In some embodiments, the subject experience greater than 2, 3, 4, or 5 immune related adverse events in response to the prior therapy.

In some embodiments, the compositions and polypeptides of the disclosure provide for a targeted delivery of a cytotoxic agent. Such targeted delivery may provide for a reduction in cardiac damage, extended survival, a reduction of the effective dose concentration, an increase in tumor-infiltrating lymphocytes, an increase in CD8⁺ cytotoxic T cells, an increase in natural killer cells, a reduction in inflammatory cytokines such as IFN-g, TNF-a, IL-5, and IL-6, or no adverse reduction of red blood cells, white blood cells, hematocrit and/or hemoglobin concentration compared to the composition comprising the same polypeptide without the collagen binding domain.

The terms “protein”, “polypeptide” and “peptide” are used interchangeably herein when referring to a gene product.

The terms “subject,” “mammal,” and “patient” are used interchangeably. In some embodiments, the subject is a mammal. In some embodiments, the subject is a human. In some embodiments, the subject is a mouse, rat, rabbit, dog, donkey, or a laboratory test animal such as fruit fly, zebrafish, etc.

In some embodiments, the patient has been previously treated for the cancer. In some embodiments, the subject was resistant to the previous cancer treatment. In some embodiments, the subject was determined to be a poor responder to the previous cancer treatment.

It is contemplated that the methods and compositions include exclusion of any of the embodiments described herein.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-H Synthesis and Characterization of Dox-CBD-SA. (A) Schematic of CBD-SA mediated drug delivery. (B) Synthesis scheme of Dox-CBD-SA. (C) Affinities (Kd values are shown) of CBD-SA and SA against collagen type I and collagen type III were measured by ELISA. N.D.=not determined due to low signal. Graphs with [concentrations] vs [signals] are shown in FIG. 7. Two experimental replicates. (D) Dox conjugation ratio per protein are presented. Values were calculated based on the results of BCA protein quantification assay (proteins) and absorbance at 495 nm (Dox) (mean±SD of three experimental replicates). (E) Dox release kinetics from Dox-CBD-SA under three different pH conditions was evaluated by fluorescence (excitation at 495 nm, emission at 590 nm) (n=3, mean±SD. Two experimental replicates). (F) MMTV-PyMT cells were seeded and incubated overnight. Dox, Dox-SA or Dox-CBD-SA were added (red). Cells were also stained with lysotracker (green). Scale bar=20 μm. Representative pictures are presented. Two experimental replicates. (G, H) Cytotoxicity of Dox variants against MMTV-PyMT cells or MC38 cells in vitro (n=6, mean±SEM). Two experimental replicates.

FIG. 2A-D Dox-CBD-SA shows comparable plasma pharmacokinetics with Dox-SA and higher tumor accumulation than aldoxorubicin and Dox-SA. (A) Aldoxorubicin, Dox-SA or Dox-CBD-SA (5 mg/kg for Dox basis) were administered to tumor-free FVB mice via tail vein injection (i.v.). Blood plasma was collected at indicated time points. Plasma concentration of Dox was measured by fluorescence (mean±SEM, n=4 for aldoxorubicin, n=5 for Dox-SA and Dox-CBD-SA). (B) Plasma half-lives of Dox were calculated using two-phase exponential decay: MFI (t)=Ae^{-αt}+Be^{-βt}. t½, α, fast clearance half-life; t½, β, slow clearance half-life. (mean±SEM. n=4 for aldoxorubicin, n=5 for Dox-SA and Dox-CBD-SA). (C) MMTV-PyMT tumor bearing mice were treated with aldoxorubicin, Dox-SA, or Dox-CBD-SA (4.16 mg/kg for Dox basis). At the indicated time points, tumors were harvested, and the amount of Dox within the tumors was quantified (mean±SEM. n=5 for 2 h, n=7 for 24 h per group). (D) 100 μg of DyLight 488-labeled SA or equimolar amounts of DyLight 488-labeled CBD-SA were injected i.v. to MMTV-PyMT tumor bearing mice. 1 h after injection, tumors were harvested and fluorescence was analyzed by confocal microscopy. Tissues were also stained with DAPI and anti-CD31 antibody. Scale bar=100 μm. Representative images of 3 tumors, each. Two experimental replicates. Statistical analyses were done using ANOVA with Tukey's test. *p<0.05; **p<0.01; N.S.=not significant.

FIG. 3A-L Dox-CBD-SA shows enhanced anti-tumor efficacy and infiltration of lymphocytes into tumor in MMTV-PyMT breast cancer model. (A) 5×10⁵ MMTV-PyMT cells were inoculated to FVB mice on day 0. Aldoxorubicin, Dox-SA, or Dox-CBD-SA (5 mg/kg for Dox basis) were injected i.v. on day 7. Graphs depict tumor volume until the first mouse died (mean±SEM). (B) Survival rate. (C-F) Individual tumor growth curves. CR indicates complete response frequency. Three experimental replicates. (G-L) 5×10⁵ MMTV-PyMT cells were inoculated on day 0. Aldoxorubicin, Dox-SA, or Dox-CBD-SA (5 mg/kg for Dox basis) were injected i.v. on day 7. Lymphocytes within tumors were extracted on day 14, followed by flow cytometric analysis. (G-I) Graphs depict the number of (G) CD45⁺CD8⁺CD3⁺ T cells, (H) CD45⁺CD4⁺CD3⁺ T cells, and (I) CD45⁺NK1.1⁺CD3⁻ NK cells per tumor weight (mg). Bars represent mean±SEM. (J-L) Graph shows [CD45⁺CD8⁺CD3⁺ T cells per tumor weight (mg)] (J), [CD45⁺CD4⁺CD3⁺ T cells per tumor weight (mg)] (K), or [CD45⁺NK1.1⁺CD3⁺ NK cells per tumor weight (mg)] (L) vs [tumor weight]. Two experimental replicates. Statistical analyses were done using (A, H, I) ANOVA with Tukey's test or (G) Kruskal-Wallis test followed by Dunn's test, or (B) Log-rank (Mantel-Cox) test. *p<0.05; **p<0.01; N.S.=not significant.

FIG. 4A-G Dox-CBD-SA treatment shows reduced toxicity. 20 mg/kg of aldoxorubicin or Dox-CBD-SA (Dox basis) were administered to tumor-free FVB mice via tail vein injection on day 0. (A-D) Plasma cytokine concentrations on day 3. (E) Red blood cell counts on day 6. (F) White blood cell counts on day 3. (G) Spleen weights on day 16. Data represents mean±SEM. Two experimental replicates. Statistical analyses were done using ANOVA with Tukey's test. *p<0.05; **p<0.01; N.S.=not significant.

FIG. 5A-H Dox-CBD-SA treatment completely eradicates established MC38 tumor in combination with anti-PD-1 checkpoint inhibitor. 5×10⁵ MC38 cells were inoculated on day 0. Mice were injected i.v. with 5 mg/kg (Dox basis) of aldoxorubicin or Dox-CBD-SA on day 6, 9, and 12. αPD-1 was also injected i.p. on day 10 and 13. (A) The experimental schedule. (B) Graphs depict tumor volume until the first mouse died (mean±SEM), CR=complete response. (C) Survival rate. (D-G) Individual tumor growth curves. CR indicates complete response frequency. (H) On day 60, Dox-CBD-SA+αPD-1 treated survivors were re-challenged by subcutaneous injection of 5×10⁵ MC38 cells. Naïve mice were also challenged with the same amounts of cells as a control group. # of mice developed palpable tumors are shown. Two experimental replicates. Statistical analyses were done using Log-rank (Mantel-Cox) test for survival curves. *p<0.05; **p<0.01; N.S.=not significant.

FIG. 6 Confirmation of CBD fusion to SA by MALDI-TOF MS analysis. CBD-SA was analyzed by MALDI-TOF MS. Abscissa is mass to charge ratio (m/z) and ordinate is intensity of charged ions. Two experimental replicates.

FIG. 7A-B Binding affinities of CBD-SA to collagen type I and III. Affinities of CBD-SA against (A) collagen type I and (B) collagen type III were determined by ELISA (n=4, mean±SD). Graphs with [concentrations] vs [signals] are shown. Two experimental replicates.

FIG. 8 SDS-PAGE analysis of mouse SA and CBD-SA conjugated with Dox. Dox-SA and Dox-CBD-SA were analyzed by SDS-PAGE with coomassie blue staining. R reduced; NR non-reduced. Representative images are presented. Two experimental replicates.

FIG. 9A-B Hydrodynamic sizes. (A) Sizes of un-conjugated CBD-SA, Dox-CBD-SA and Dox-CBD-SA reconstituted after lyophilization were measured by DLS. (B) Sizes of un-conjugated SA and Dox-SA were also measured. Two experimental replicates.

FIG. 10 The binding interface between collagen type III and A3 domain of von Willebrand factor. Crystal structure of the A3 domain of von Willebrand factor (CBD) in complex with type III collagen (PDB 4DMU). The Image was processed using UCSF chimera. Lysines are indicated as blue color.

FIG. 11 In vitro release kinetics of Dox from Dox-SA. Dox release kinetics from Dox-SA under three different pH conditions were evaluated by fluorescence (excitation at 495 nm, emission at 590 nm, n=3, mean±SD). Two experimental replicates.

FIG. 12A-B Plasma pharmacokinetics of DyLight 800 labeled SA and CBD-SA. 200 μg of DyLight 800 labeled SA or CBD-SA were administered to tumor-free FVB mice via tail vein injection (i.v.). Blood plasma was collected at indicated time points. (A) Signal intensity of each sample was normalized with mean signal intensity of samples collected at 1 min after injection (mean±SEM, n=4). (B) Plasma half-lives of labeled SA and CBD-SA were calculated using two-phase exponential decay: MFI (t)=A^{e-t}+Be^{-βt}, t½, β, slow clearance half-life. (mean±SEM. n=4). One experimental replicate.

FIG. 13A-B Changes of hematological values in mice receiving 20 mg/kg of aldoxorubicin or Dox-CBD-SA. (A) Hematocrit and (B) hemoglobin concentration on day 6 after injection. Two experimental replicates. Statistical analyses were done using ANOVA with Tukey's test. **p<0.01; N.S.=not significant.

FIG. 14 Histological analysis of major organs after Dox-CBD-SA treatment. Tumor-free FVB mice received Dox-CBD-SA (20 mg/kg) on day 0. On day 16, heart, liver, kidney, and lung were harvested and processed to obtain histologic sections (n=7 for untreated, n=5 for Dox-CBD-SA). Scale bar=200 μm. H&E stained histology was evaluated blindly and no significant abnormality was observed after Dox-CBD-SA treatment. Representative images are shown. Two experimental replicates.

FIG. 15A-B MC38 tumor re-challenge and body weight changes of MC38 tumor-bearing mice during the treatment. (A) Graph depicts tumor sizes of Dox-CBD-SA+αPD-1 treated survivors re-challenged with MC38 cells (mean±SEM). Naïve mice were also challenged with the same amounts of cells as a control group. # of mice developed a palpable tumor is shown. (B) Body weight changes of mice during the treatments in FIG. 5 (mean±SEM). A line represents 85% of initial body weight. Two experimental replicates.

### DETAILED DESCRIPTION

Because small molecule anticancer drugs broadly distribute to tissues and induce systemic side effects, modifications of drugs to improve their pharmacokinetics and bio-distribution have been attempted. Active targeting of tumor-specific or tumor-associated antigens for drug delivery is a therapeutic strategy. However, this intrinsically limits the applicable range of cancers and may also lead to acquired drug resistance due to antigen-selective cell targeting and killing, which antigen may be lost by mutation. Here, the inventors engineered CBD-SA (collagen binding domain-serum albumin) to overcome these issues. Unlike other active targeting strategies, CBD-SA does not require the prior investigation of tumor-associated antigen expression, because collagen is nearly ubiquitously expressed in tumors, and the CBD gains access to the tumor stroma via the abnormal blood vessel structure within the tumor microenvironment. Subsequently, the CBD-SA binds to exposed collagen and converts the tumor stroma into a reservoir for chemotherapeutics.

Cardiac toxicity is a major drawback of Dox, which limits the lifetime cumulative dose of Dox. Surprisingly, it was found that even 20 mg/kg of Dox-CBD-SA administration did not show any signs of cardiac damage. This suggests that Dox pre-conjugated with CBD-SA is less cardiotoxic than free Dox, which irreversibly damages cardiac tissue at a cumulative dose of 15 mg/kg in mouse models. Importantly, a cumulative dose of 15 mg/kg is nearly equivalent to the maximum cumulative dose in human. The efficacy and non-specific toxicity of the conjugates are somewhat surprising and unexpected, since one may hypothesize that CBD-SA might accumulate in undesirable sites in the body such as liver, kidney, and wounds, where collagens may be exposed via a fenestrated or leaky endothelium. However, the inventors did not observe pathological damage in the liver and kidney after 20 mg/kg of Dox-CBD-SA administration. Thus, the current disclosure describes a novel strategy for targeting cytotoxic agents to the tumor environment.

### I. Targeting Polypeptide

A. Collagen Binding Domain

von Willebrand factor (vWF) is a blood coagulation factor and binds to both type I and type III collagen, and the adhesion receptor GPIb on blood platelets. When injured, collagen beneath endothelial cells is exposed to blood plasma, and vWF-collagen binding initiates the thrombosis cascade. The vWF A domain has the highest affinity against collagen among reported non-bacterial origin proteins/peptides.

In some embodiments, the polypeptide comprises a collagen binding domain from decorin. In some embodiments, the collagen binding domain comprises a decorin peptide such as LRELHLNNNC (SEQ ID NO:11), which is derived from bovine or LRELHLDNNC (SEQ ID NO:12), which is derived from human.

In some embodiments, the collagen binding domain comprises a peptide fragment from human decorin, which is represented by the following amino acid sequence:

(SEQ ID NO: 13)
CGPFQQRGLFDFMLEDEASGIGPEVPDDRDFEPSLGPVCPFRCQCHLRVV
QCSDLGLDKVPKDLPPDTTLLDLQNNKITEIKDGDFKNLKNLHALILVNN
KISKVSPGAFTPLVKLERLYLSKNQLKELPEKMPKTLQELRAHENEITKV
RKVTFNGLNQMIVIELGTNPLKSSGIENGAFQGMKKLSYIRIADTNITSI
PQGLPPSLTELHLDGNKISRVDAASLKGLNNLAKLGLSFNSISAVDNGSL
ANTPHLRELHLDNNKLTRVPGGLAEHKYIQVVYLHNNNISVVGSSDFCPP
GHNTKKASYSGVSLFSNPVQYWEIQPSTFRCVYVRSAIQLGNYK.

In some embodiments, the collagen binding peptide is a peptide from von Willebrand factor (vWF). The sequence of human vWF comprises the following:

(SEQ ID NO: 4)
MIPARFAGVLLALALILPGTLCAEGTRGRSSTARCSLFGSDFVNTFDGSM
YSFAGYCSYLLAGGCQKRSFSIIGDFQNGKRVSLSVYLGEFFDIHLFVNG
TVTQGDQRVSMPYASKGLYLETEAGYYKLSGEAYGFVARIDGSGNFQVLL
SDRYFNKTCGLCGNENIFAEDDFMTQEGTLTSDPYDFANSWALSSGEQWC
ERASPPSSSCNISSGEMQKGLWEQCQLLKSTSVFARCHPLVDPEPFVALC
EKTLCECAGGLECACPALLEYARTCAQEGMVLYGWTDHSACSPVCPAGME
YRQCVSPCARTCQSLHINEMCQERCVDGCSCPEGQLLDEGLCVESTECPC
VHSGKRYPPGTSLSRDCNTCICRNSQWICSNEECPGECLVTGQSHFKSFD
NRYFTFSGICQYLLARDCQDHSFSIVIETVQCADDRDAVCTRSVTVRLPG
LHNSLVKLKHGAGVAMDGQDVQLPLLKGDLRIQHTVTASVRLSYGEDLQM
DWDGRGRLLVKLSPVYAGKTCGLCGNYNGNQGDDFLTPSGLAEPRVEDFG
NAWKLHGDCQDLQKQHSDPCALNPRMTRFSEEACAVLTSPTFEACHRAVS
PLPYLRNCRYDVCSCSDGRECLCGALASYAAACAGRGVRVAWREPGRCEL
NCPKGQVYLQCGTPCNLTCRSLSYPDEECNEACLEGCFCPPGLYMDERGD
CVPKAQCPCYYDGEIFQPEDIFSDHHTMCYCEDGFMHCTMSGVPGSLLPD
AVLSSPLSHRSKRSLSCRPPMVKLVCPADNLRAEGLECTKTCQNYDLECM
SMGCVSGCLCPPGMVRHENRCVALERCPCFHQGKEYAPGETVKIGCNTCV
CRDRKWNCTDHVCDATCSTIGMAHYLTEDGLKYLFPGECQYVLVQDYCGS
NPGTFRILVGNKGCSHPSVKCKKRVTILVEGGEIELFDGEVNVKRPMKDE
THFEVVESGRYIILLLGKALSVVWDRHLSISVVLKQTYQEKVCGLCGNFD
GIQNNDLTSSNLQVEEDPVDFGNSWKVSSQCADTRKVPLDSSPATCHNNI
MKQTMVDSSCRILTSDVFQDCNKLVDPEPYLDVCIYDTCSCESIGDCACF
CDTIAAYAHVCAQHGKVVTWRTATLCPQSCEERNLRENGYECEWRYNSCA
PACQVTCQHPEPLACPVQCVEGCHAHCPPGKILDELLQTCVDPEDCPVCE
VAGRRFASGKKVTLNPSDPEHCQICHCDVVNLTCEACQEPGGLVVPPTDA
PVSPTTLYVEDISEPPLHDFYCSRLLDLVFLLDGSSRLSEAEFEVLKAFV
VDMMERLRISQKWVRVAVVEYHDGSHAYIGLKDRKRPSELRRIASQVKYA
GSQVASTSEVLKYTLFQIFSKIDRPEASRITLLLMASQEPQRMSRNFVRY
VQGLKKKKVIVIPVGIGPHANLKQIRLIEKQAPENKAFVLSSVDELEQQR
DEIVSYLCDLAPEAPPPTLPPDMAQVTVGPGLLGVSTLGPKRNSMVLDVA
FVLEGSDKIGEADENRSKEEMEEVIQRMDVGQDSIHVTVLQYSYMVTVEY
PFSEAQSKGDILQRVREIRYQGGNRTNTGLALRYLSDHSFLVSQGDREQA
PNLVYMVTGNPASDEIKRLPGDIQVVPIGVGPNANVQELERIGWPNAPIL
IQDFETLPREAPDLVLQRCCSGEGLQIPTLSPAPDCSQPLDVILLLDGSS
SFPASYFDEMKSFAKAFISKANIGPRLTQVSVLQYGSITTIDVPWNVVPE
KAHLLSLVDVMQREGGPSQIGDALGFAVRYLTSEMHGARPGASKAVVILV
TDVSVDSVDAAADAARSNRVTVFPIGIGDRYDAAQLRILAGPAGDSNVVK
LQRIEDLPTMVTLGNSFLHKLCSGFVRICMDEDGNEKRPGDVWTLPDQCH
TVTCQPDGQTLLKSHRVNCDRGLRPSCPNSQSPVKVEETCGCRWTCPCVC
TGSSTRHIVTFDGQNFKLTGSCSYVLFQNKEQDLEVILHNGACSPGARQG
CMKSIEVKHSALSVELHSDMEVTVNGRLVSVPYVGGNMEVNVYGAIMHEV
RFNHLGHIFTFTPQNNEFQLQLSPKTFASKTYGLCGICDENGANDFMLRD
GTVTTDWKTLVQEWTVQRPGQTCQPILEEQCLVPDSSHCQVLLLPLFAEC
HKVLAPATFYAICQQDSCHQEQVCEVIASYAHLCRTNGVCVDWRTPDFCA
MSCPPSLVYNHCEHGCPRHCDGNVSSCGDHPSEGCFCPPDKVMLEGSCVP
EEACTQCIGEDGVQHQFLEAWVPDHQPCQICTCLSGRKVNCTTQPCPTAK
APTCGLCEVARLRQNADQCCPEYECVCDPVSCDLPPVPHCERGLQPTLTN
PGECRPNFTCACRKEECKRVSPPSCPPHRLPTLRKTQCCDEYECACNCVN
STVSCPLGYLASTATNDCGCTTTTCLPDKVCVHRSTIYPVGQFWEEGCDV
CTCTDMEDAVMGLRVAQCSQKPCEDSCRSGFTYVLHEGECCGRCLPSACE
VVTGSPRGDSQSSWKSVGSQWASPENPCLINECVRVKEEVFIQQRNVSCP
QLEVPVCPSGFQLSCKTSACCPSCRCERMEACMLNGTVIGPGKTVMIDVC
TTCRCMVQVGVISGFKLECRKTTCNPCPLGYKEENNTGECCGRCLPTACT
IQLRGGQINITLKRDETLQDGCDTHFCKVNERGEYFWEKRVTGCPPFDEH
KCLAEGGKIMKIPGTCCDTCEEPECNDITARLQYVKVGSCKSEVEVDIHY
CQGKCASKAMYSIDINDVQDQCSCCSPTRTEPMQVALHCTNGSVVYHEVL
NAMECKCSPRKCSK.

In some embodiments, the peptide is from the vWF A3 domain. The VWF A3 domain is derived from the human sequence, residues 1670-1874 (907-1111 of mature VWF) and has the following sequence:

(SEQ ID NO: 1)
CSGEGLQIPTLSPAPDCSQPLDVILLLDGSSSFPASYFDEMKSFAKAFIS
KANIGPRLTQVSVLQYGSITTIDVPWNVVPEKAHLLSLVDVMQREGGPSQ
IGDALGFAVRYLTSEMEIGARPGASKAVVILVTDVSVDSVDAAADAARSN
RVTVFPIGIGDRYDAAQLRILAGPAGDSNVVKLQRIEDLPTMVTLGNSFL
HKLCSG.

In some embodiments, the ECM-peptide comprises all or a fragment of vWF A3, which is represented by the following amino acid sequences:

(SEQ ID NO: 14)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRICTG.

In some embodiments, the collagen binding domain comprises a polypeptide with the following sequence:

(SEQ ID NO: 2)
CSQPLDVILLLDGSSSFPASYFDEMKSFAKAFISKANIGPRLTQVSVL
QYGSITTIDVPWNVVPEKAHLLSLVDVMQREGGPSQIGDALGFAVRYL
TSEMHGARPGASKAVVILVTDVSVDSVDAAADAARSNRVTVFPIGIGD
RYDAAQLRILAGPAGDSNVVKLQRIEDLPTMVTLGNSFLHKLCSGFVR
I

In some embodiments, the polypeptide comprises a collagen binding domain albumin polypeptide having the following sequence:

(SEQ ID NO: 3)
CSQPLDVILLLDGSSSFPASYFDEMKSFAKAFISKANIGPRLTQVSVL
QYGSITTIDVPWNVVPEKAHLLSLVDVMQREGGPSQIGDALGFAVRYL
TSEMHGARPGASKAVVILVTDVSVDSVDAAADAARSNRVTVFPIGIGD
RYDAAQLRILAGPAGDSNVVKLQRIEDLPTMVTLGNSFLHKLCSGFVR
IGGGSGGGSEAHKSEIAHRYNDLGEQHFKGLVLIAFSQYLQKCSYDEH
AKLVQEVTDFAKTCVADESAANCDKSLHTLFGDKLCAIPNLRENYGEL
ADCCTKQEPERNECFLQHKDDNPSLPPFERPEAEAMCTSFKENPTTFM
GHYLHEVARRHPYFYAPELLYYAEQYNEILTQCCAEADKESCLTPKLD
GVKEKALVSSVRQRMKCSSMQKFGERAFKAWAVARLSQTFPNADFAEI
TKLATDLTKVNKECCHGDLLECADDRAELAKYMCENQATISSKLQTCC
DKPLLKKAHCLSEVEHDTMPADLPAIAADFVEDQEVCKNYAEAKDVFL
GTFLYEYSRRHPDYSVSLLLRLAKKYEATLEKCCAEANPPACYGTVLA
EFQPLVEEPKNLVKTNCDLYEKLGEYGFQNAILVRYTQKAPQVSTPTL
VEAARNLGRVGTKCCTLPEDQRLPCVEDYLSAILNRVCLLHEKTPVSE
HVTKCCSGSLVERRPCFSALTVDETYVPKEFKAETFTFHSDICTLPEK
EKQIKKQTALAELVKHKPKATAEQLKTVMDDFAQFLDTCCKAADKDTC
FSTEGPNLVTRCKDALAHHHHHH

Exemplary peptides include all or part of any one of SEQ ID NO:1-4 or 11-14. The collagen binding domain may be a polypeptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of the disclosure, such as to SEQ ID NO: 1-4 or 11-14.

B. Linker

A linker sequence may be included in the polypeptides. For example, a linker having at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids (or any derivable range therein) may separate that antibody and the peptide.

In some embodiments, the albumin polypeptide, IgG Fc domain polypeptide, collagen binding domain and/or cytotoxic agent are covalently linked. For example, the cytotoxic agent may be covalently linked to the collagen binding domain. In some embodiments, the cytotoxic agent is covalently linked to the albumin polypeptide. In some embodiments, the cytotoxic agent is covalently linked to the IgG Fc domain polypeptide. In some embodiments, a linker is between the cytotoxic agent and the collagen binding domain or the albumin polypeptide. In some embodiments, a linker is between the cytotoxic agent and the collagen binding domain or the IgG Fc domain polypeptide. In some embodiments, the albumin polypeptide is covalently linked to the collagen domain. In some embodiments, the IgG Fc domain polypeptide is covalently linked to the collagen domain. In some embodiments, a linker is between the albumin polypeptide and the collagen binding domain. In some embodiments, a linker is between the IgG Fc domain polypeptide and the collagen binding domain. In some embodiments, the linker comprises a bifunctional linker. Linkers, such as amino acid or peptidomimetic sequences may be inserted between the peptide and/or antibody sequence. Linkers may have one or more properties that include a flexible conformation, an inability to form an ordered secondary structure or a hydrophobic or charged character which could promote or interact with either domain. Examples of amino acids typically found in flexible protein regions may include Gly, Asn and Ser. For example, a suitable peptide linker may be GGGSGGGS (SEQ ID NO:5) or (GGGS)n (SEQ ID NO:6), wherein n=1, 2, 3, 4, 5, 6, 7, 8, 9, or 10 (or any range derivable therein). Other near neutral amino acids, such as Thr and Ala, may also be used in the linker sequence. The length of the linker sequence may vary without significantly affecting the function or activity of the fusion protein (see, e.g., U.S. Pat. No. 6,087,329). In a particular aspect, the linker may be at least, at most, or exactly 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acid residues (or any range derivable therein). Examples of linkers may also include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST). Examples of linkers further comprise a linear carbon chain, such as C_{N} (where N=1-100 carbon atoms). In some embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. In some embodiments, the linker is sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its sulfo-NHS ester is reactive toward primary amines (as found in lysine and the protein or peptide N-terminus). Further, the linker may be maleimidocaproyl (mc). In some embodiments, the covalent linkage may be achieved through the use of Traut's reagent.

C. Albumin

In some embodiments, the albumin polypeptide is from mouse. In some embodiments, the albumin polypeptide is from humans.

In some embodiments, the albumin polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence:

(SEQ ID NO: 7)
MKWVTFISLLFLFSSAYSRGVERRDAHKSEVAHRFKDLGEENFKALVL
IAFAQYLQQCPFEDHVKLVNEVTEFAKTCVADESAENCDKSLHTLFGD
KLCTVATLRETYGEMADCCAKQEPERNECFLQHKDDNPNLPRLVRPEV
DVMCTAFHDNEETFLKKYLYEIARRHPYFYAPELLFFAKRYKAAFTEC
CQAADKAACLLPKLDELRDEGKASSAKQRLKCASLQKFGERAFKAWAV
ARLSQRFPKAEFAEVSKLVTDLTKVHTECCHGDLLECADDRADLAKYI
CENQDSISSKLKECCEKPLLEKSHCIAEVENDEMPADLPSLAADFVES
KDVCKNYAEAKDVFLGMFLYEYARRHPDYSVVLLLRLAKTYKTTLEKC
CAAADPHECYAKVFDEFKPLVEEPQNLIKQNCELFEQLGEYKFQNALL
VRYTKKVPQVSTPTLVEVSRNLGKVGSKCCKHPEAKRMPCAEDYLSVV
LNQLCVLHEKTPVSDRVTKCCTESLVNRRPCFSALEVDETYVPKEFNA
ETFTFHADICTLSEKERQIKKQTALVELVKHKPKATKEQLKAVMDDFA
AFVEKCCKADDKETCFAEEGKKLVAASRAALGL.

In some embodiments, the albumin polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence:

(SEQ ID NO: 8)
EAHKSEIAHRYNDLGEQHFKGLVLIAFSQYLQKCSYDEHAKLVQEVTD
FAKTCVADESAANCDKSLHTLFGDKLCAIPNLRENYGELADCCTKQEP
ERNECFLQHKDDNPSLPPFERPEAEAMCTSFKENPTTFMGHYLHEVAR
RHPYFYAPELLYYAEQYNEILTQCCAEADKESCLTPKLDGVKEKALVS
SVRQRMKCSSMQKFGERAFKAWAVARLSQTFPNADFAEITKLATDLTK
VNKECCHGDLLECADDRAELAKYMCENQATISSKLQTCCDKPLLKKAH
CLSEVEHDTMPADLPAIAADFVEDQEVCKNYAEAKDVFLGTFLYEYSR
RHPDYSVSLLLRLAKKYEATLEKCCAEANPPACYGTVLAEFQPLVEEP
KNLVKTNCDLYEKLGEYGFQNAILVRYTQKAPQVSTPTLVEAARNLGR
VGTKCCTLPEDQRLPCVEDYLSAILNRVCLLHEKTPVSEHVTKCCSGS
LVERRPCFSALTVDETYVPKEFKAETFTFHSDICTLPEKEKQIKKQTA
LAELVKHKPKATAEQLKTVMDDFAQFLDTCCKAADKDTCFSTEGPNLV
TRCKDALA.

In some embodiments, the albumin polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence:

(SEQ ID NO: 9)
MKWVTFLLLLFVSGSAFSRGVERREAHKSEIAHRYNDLGEQHFKGLVL
IAFSQYLQKCSYDEHAKLVQEVTDFAKTCVADESAANCDKSLHTLFGD
KLCAIPNLRENYGELADCCTKQEPERNECFLQHKDDNPSLPPFERPEA
EAMCTSFKENPTTFMGHYLHEVARRHPYFYAPELLYYAEQYNEILTQC
CAEADKESCLTPKLDGVKEKALVSSVRQRMKCSSMQKFGERAFKAWAV
ARLSQTFPNADFAEITKLATDLTKVNKECCHGDLLECADDRAELAKYM
CENQATISSKLQTCCDKPLLKKAHCLSEVEHDTMPADLPAIAADFVED
QEVCKNYAEAKDVFLGTFLYEYSRRHPDYSVSLLLRLAKKYEATLEKC
CAEANPPACYGTVLAEFQPLVEEPKNLVKTNCDLYEKLGEYGFQNAIL
VRYTQKAPQVSTPTLVEAARNLGRVGTKCCTLPEDQRLPCVEDYLSAI
LNRVCLLHEKTPVSEHVTKCCSGSLVERRPCFSALTVDETYVPKEFKA
ETFTFHSDICTLPEKEKQIKKQTALAELVKHKPKATAEQLKTVMDDFA
QFLDTCCKAADKDTCFSTEGPNLVTRCKDALA.

In some embodiments, the albumin polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence:

(SEQ ID NO: 10)
DAHKSEVAHREKDLGEENFKALVLIAFAQYLQQCPFEDHVKLVNEVTE
FAKTCVADESAENCDKSLHTLFGDKLCTVATLRETYGEMADCCAKQEP
ERNECFLQHKDDNPNLPRLVRPEVDVMCTAFHDNEETFLKKYLYEIAR
RHPYFYAPELLFFAKRYKAAFTECCQAADKAACLLPKLDELRDEGKAS
SAKQRLKCASLQKFGERAFKAWAVARLSQRFPKAEFAEVSKLVTDLTK
VHTECCHGDLLECADDRADLAKYICENQDSISSKLKECCEKPLLEKSH
CIAEVENDEMPADLPSLAADFVESKDVCKNYAEAKDVFLGMFLYEYAR
RHPDYSVVLLLRLAKTYKTTLEKCCAAADPHECYAKVEDEFKPLVEEP
QNLIKQNCELFEQLGEYKFQNALLVRYTKKVPQVSTPTLVEVSRNLGK
VGSKCCKHPEAKRMPCAEDYLSVVLNQLCVLHEKTPVSDRVTKCCTES
LVNRRPCFSALEVDETYVPKEFNAETFTFHADICTLSEKERQIKKQTA
LVELVKHKPKATKEQLKAVMDDFAAFVEKCCKADDKETCFAEEGKKLV
AASRAALG.

D. Fc Domain from Human IgG

Similar to albumin, Fc domain from human IgG is used to enhance drug half-life, because Fc domain also has cell recycling system as with albumin. In some embodiments, the albumin polypeptide is from humans.

In some embodiments, the hIgG1 Fc polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence (IGHG1, 99-330):

(SEQ ID NO: 15)
EPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMT
KNQVSLTCLVKGFYPSDIAVEWESNGQPENNYKTTPPVLDSDGSFFLY
SKLTVDKSRWQQGNVFSCSVMHEALHNHYTQKSLSLSPGK.

In some embodiments, the hIgG2 Fc polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence (IGHG2, 99-326):

(SEQ ID NO: 16)
ERKCCVECPPCPAPPVAGPSVFLFPPKPKDTLMISRTPEVTCVVVDVS
HEDPEVQFNWYVDGMEVHNAKTKPREEQFNSTFRVVSVLTVVHQDWLN
GKEYKCKVSNKGLPAPIEKTISKTKGQPREPQVYTLPPSREEMTKNQV
SLTCLVKGFYPSDIAVEWESNGQPENNYKTTPPMLDSDGSFFLYSKLT
VDKSRWQQGNVFSCSVMHEALHNHYTQKSLSLSPGK.

In some embodiments, the hIgG3 Fc polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence (IGHG3, 99-376):

(SEQ ID NO: 17)
ELKTPLGDTTHTCPRCPEPKSCDTPPPCPRCPEPKSCDTPPPCPRCPE
PKSCDTPPPCPRCPAPELLGGPSVFLEPPKPKDTLMISRTPEVTCVVV
DVSHEDPEVQFKWYVDGVEVHNAKTKPREEQYNSTFRVVSVLTVLHQD
WLNGKEYKCKVSNKALPAPIEKTISKTKGQPREPQVYTLPPSREEMTK
NQVSLTCLVKGFYPSDIAVEWESSGQPENNYNTTPPMLDSDGSFFLYS
KLTVDKSRWQQGNIFSCSVMHEALHNRFTQKSLSLSPGK.

In some embodiments, the hIgG4 Fc polypeptide may comprise a polypeptide or fragment with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide having the following sequence (IGHG4, 99-327):

(SEQ ID NO: 18)
ESKYGPPCPSCPAPEFLGGPSVFLFPPKPKDTLMISRTPEVTCVVVDV
SQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRL
TVDKSRWQEGNVFSCSVMHEALHNHYTQKSLSLSLGK.

### II. Cytotoxic Agents

Embodiments of the disclosure relate to albumin-collagen binding domain conjugates linked to cytotoxic agents. Embodiments of the disclosure relate to IgG Fc domain-collagen binding domain conjugates linked to cytotoxic agents. Cytotoxic agents include the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins, dolastatins, maytansinoids, differentiation inducers, and taxols.

Members of these classes include, for example, taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin and podophyllotoxin derivatives such as etoposide or etoposide phosphate, vinblastine, vincristine, vindesine, taxanes including taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, maytansinoids (including DM1), monomethylauristatin E (MMAE), monomethylauristatin F (MMAF), and maytansinoids (DM4) and their analogues.

Cytotoxic agents also include bacterial toxins such as diphtheria toxin, plant toxins such as ricin, small molecule toxins such as geldanamycin, maytansinoids, and calicheamicin, hemiasterlins. Toxins may exert their cytotoxic and cytostatic effects by mechanisms including tubulin binding, DNA binding, or topoisomerase inhibition.

Cytotoxic agents, such as a maytansinoids, dolastatins, auristatins, a trichothecene, calicheamicin, and CC1065, and the derivatives of these toxins that have toxin activity, may also be used. Other cytotoxic agents include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296). Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993. In some embodiments, the cytotoxic agent comprises a chemotherapeutic described herein.

### III. Additional Therapies

A. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

1. Checkpoint Inhibitors and Combination Treatment

Embodiments of the disclosure may include administration of immune checkpoint inhibitors (also referred to as checkpoint inhibitor therapy), which are further described below.

a. PD-1, PD-L1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect, the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PD-L1, or PD-L2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD4OLG), GITR (TNFRSF18), and combinations thereof. Inhibitors include inhibitory antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

6. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

B. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

C. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some embodiments, the additional therapy comprises neoantigen administration. Many tumors express mutations. These mutations potentially create new targetable antigens (neoantigens) for use in T cell immunotherapy. The presence of CD8⁺ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. Nucleic Acids

In certain embodiments, there are recombinant nucleic acids encoding the polypeptides described herein.

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or fewer in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein (see above).

In particular embodiments, there are isolated nucleic acid segments and recombinant vectors incorporating nucleic acid sequences that encode a polypeptides (e.g., a polymerase, RNA polymerase, one or more truncated polymerase domains or interaction components that are polypeptides) that drive gene transcription dependent on polymerase activity from the polymerase domains when the interaction components interact. The term “recombinant” may be used in conjunction with a polypeptide or the name of a specific polypeptide, and this generally refers to a polypeptide produced from a nucleic acid molecule that has been manipulated in vitro or that is a replication product of such a molecule.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

A. Vectors

Polypeptides may be encoded by a nucleic acid molecule. The nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce a polymerase, RNA polymerase, one or more truncated polymerase domains or interaction components that are fused, attached or linked to the one or more truncated RNA polymerase domains.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described herein.

B. Cells

The disclosure provides methods for modifying a target RNA of interest, in particular in prokaryotic cells, eukaryotic cells, tissues, organs, or organisms, more in particular in mammalian cells, tissues, organs, or organisms. The target RNA may be comprised in a nucleic acid molecule within a cell. In some embodiments, the target RNA is in a eukaryotic cell, such as a mammalian cell or a plant cell. The mammalian cell many be a human, non-human primate, bovine, porcine, rodent or mouse cell. The cell may be a non-mammalian eukaryotic cell such as poultry, fish or shrimp. The plant cell may be of a crop plant such as cassava, corn, sorghum, wheat, or rice. The plant cell may also be of an algae, tree or vegetable. The modulation of the RNA induced in the cell by the methods, systems, and compositions of the disclosure may be such that the cell and progeny of the cell are altered for improved production of biologic products such as an antibody, starch, alcohol or other desired cellular output. The modulation of the RNA induced in the cell may be such that the cell and progeny of the cell include an alteration that changes the biologic product produced.

The mammalian cell may be a human or non-human mammal, e.g., primate, bovine, ovine, porcine, canine, rodent, Leporidae such as monkey, cow, sheep, pig, dog, rabbit, rat or mouse cell. The cell may be a non-mammalian eukaryotic cell such as poultry bird (e.g., chicken), vertebrate fish (e.g., salmon) or shellfish (e.g., oyster, clam, lobster, shrimp) cell. The cell may also be a plant cell. The plant cell may be of a monocot or dicot or of a crop or grain plant such as cassava, com, sorghum, soybean, wheat, oat or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.).

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with an embodiment to produce nucleic acid sequences, or their cognate polypeptides, proteins and peptides. For example, the vectors, fusion proteins, RNA hairpin binding proteins, RNA targeting molecules, RNA regulatory domain, and accessory proteins of the disclosure may utilize an expression system, such as an inducible or constitutive expression system. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems, other examples of expression systems include STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### V. Proteinaceous Compositions

The polypeptides or polynucleotides of the disclosure such as those comprising or encoding for an albumin polypeptide linked to a collagen binding domain, may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000, 1500, or 2000 or more contiguous amino acids or nucleic acids, or any range derivable therein, of any of SEQ ID NOs:1-18.

The polypeptides or polynucleotides of the disclosure such as those comprising or encoding for an albumin polypeptide linked to a collagen binding domain, may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000, 1500, 2000 or more contiguous amino acids, or any range derivable therein, of any of SEQ ID NO:1-18.

In some embodiments, the polypeptide comprises amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 650, 700, 750, 800, 850, 900, 1000, 1500, or 2000 (or any derivable range therein) of SEQ ID NOs:1-18.

In some embodiments, the polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 650, 700, 750, 800, 850, 900, 1000, 1500, or 2000 (or any derivable range therein) contiguous amino acids of any of SEQ ID NOs:1-18.

In some embodiments, the polypeptide comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 650, 700, 750, 800, 850, 900, 1000, 1500, or 2000 (or any derivable range therein) contiguous amino acids of any of SEQ ID NOs:1-18 and starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 650, 700, 750, 800, 850, 900, 1000, 1500, or 2000 of any of SEQ ID NO:1-18.

In some embodiments, the polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 650, 700, 750, 800, 850, 900, 1000, 1500, or 2000 (or any derivable range therein) contiguous amino acids of SEQ ID NOs:1-18 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of any of SEQ ID NOS:1-18.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position or nucleic acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 650, 700, 750, 800, 850, 900, 1000, 1500, or 2000 of any of SEQ ID NO:1-18 (or any derivable range therein).

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### VI. Combination Therapy

The compositions and related methods of the present disclosure, particularly administration of a polypeptide comprising an albumin polypeptide or IgG Fc domain polypeptide linked to a collagen binding domain may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

Various combination regimens of the therapeutic agents and treatments may be employed. Non-limiting examples of such combinations are shown below, wherein a therapeutic agent such as a composition disclosed herein is “A” and a second agent, such as an additional agent, chemotherapeutic, or checkpoint inhibitor described herein or known in the art is “B”.

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In some embodiments, more than one course of therapy may be employed. It is contemplated that multiple courses may be implemented.

### VII. Therapeutic Methods

The current methods and compositions relate to methods for treating cancer. In some embodiments, the cancer comprises a solid tumor. In some embodiments, the cancer is non-lymphatic. In some embodiments, the cancer is breast cancer or colon cancer.

The compositions of the disclosure may be used for in vivo, in vitro, or ex vivo administration. The route of administration of the composition may be, for example, intratumoral, intracutaneous, subcutaneous, intravenous, intralymphatic, and intraperitoneal administrations. In some embodiments, the administration is intratumoral or intralymphatic or peri-tumoral. In some embodiments, the compositions are administered directly into a cancer tissue or a lymph node.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The cancers amenable for treatment include, but are not limited to, tumors of all types, locations, sizes, and characteristics. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, specific breast cancers such as ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, paget's disease of the nipple, phyllodes tumors of the breast, recurrent and/or metastatic breast, cancer, luminal A or B breast cancer, triple-negative/basal-like breast cancer, and HER2-enriched breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer , leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous hi stiocytoma of bone/osteosarcoma, childhood medulloblastoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### VIII. Pharmaceutical Compositions and Methods

In some embodiments, pharmaceutical compositions are administered to a subject. Different aspects involve administering an effective amount of a composition to a subject. In some embodiments, a composition comprising an inhibitor may be administered to the subject or patient to treat cancer or reduce the size of a tumor. Additionally, such compounds can be administered in combination with an additional cancer therapy.

Compositions can be formulated for parenteral administration, e.g., formulated for injection via the intravenous, transcatheter injection, intraarterial injection, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. The preparation of such formulations will be known to those of skill in the art in light of the present disclosure. Other routes of administration include intratumoral, peri-tumoral, intralymphatic, injection into cancer tissue, and injection into lymph nodes. In some embodiments, the administration is systemic.

Other routes of administration are also contemplated. For example, the constructs and agents may be administered in association with a carrier. In some embodiments, the carrier is a nanoparticle or microparticle. In some embodiments, the nanoparticle or microparticle is a tumor directed nanoparticle or microparticle. For example, the carrier may further comprise a targeting moiety that directs the carrier to the tumor. The targeting moiety may be a binding agent (e.g. antibody, including scFv, etc. or other antigen binding agent) that specifically recognizes tumor cells. In some embodiments, the construct is enclosed within the carrier. In some embodiments, the construct is covalently or non-covalently attached to the surface of the carrier. In some embodiments, the carrier is a liposome. In further embodiments, a carrier molecule described herein is excluded.

Particles can have a structure of variable dimension and known variously as a microsphere, microparticle, nanoparticle, nanosphere, or liposome. Such particulate formulations can be formed by covalent or non-covalent coupling of the construct to the particle. In some embodiments, particles described herein are excluded.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after symptoms of an infection have disappeared or been reduced.

### IX. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Engineered Collagen-Binding Serum Albumin as a Drug-Conjugate Carrier for Cancer Therapy

Serum albumin (SA) is used as a carrier to deliver cytotoxic agents to tumors via passive targeting. To further improve SA's tumor targeting capacity, the inventors sought to develop an approach to retain SA-drug conjugates within tumors through a combination of passive and active targeting. SA was recombinantly fused with a collagen binding domain (CBD) of von Willebrand factor to bind within the tumor stroma after extravasation due to tumor vascular permeability. doxorubicin (Dox) was conjugated to the CBD-SA via a pH-sensitive linker. Dox-CBD-SA treatment significantly suppressed tumor growth compared to both Dox-SA and aldoxorubicin treatment in a mouse model of breast cancer. Dox-CBD-SA efficiently stimulated host anti-tumor immunity, resulting in the complete eradication of MC38 colon carcinoma when used in combination with anti-PD-1 checkpoint inhibitor. Dox-CBD-SA decreased adverse events compared to aldoxorubicin. Thus, engineered CBD-SA could be a versatile and clinically-relevant drug conjugate carrier protein for treatment of solid tumors.

A. Introduction

Doxorubicin (Dox) is a small molecule anticancer drug that is approved for treating a broad spectrum of cancers by the US Food and Drug Administration (FDA). Dox internalizes within cells via passive transmembrane diffusion and interferes with DNA functions, leading to death of proliferating cells. Although Dox treatment prolongs survival of some populations of patients, anti-tumor efficacy is not dramatic partially due to acquired drug resistance. The poor therapeutic index of Dox also limits its therapeutic use. Indeed, considerable toxicity of Dox has been reported in the clinic, including bone marrow suppression, excessive inflammation, and cardiotoxicity (13, 14). To improve efficacy, Dox is often used in combination with other chemotherapeutic agents. Here the inventors designed recombinant mouse SA (CBD-SA) in which the N-terminus is fused with the C-terminus of the VWF A3 domain, and aldoxorubicin was conjugated to CBD-SA via a pH-dependent cleavable hydrazone linkage prior to injection (namely, Dox-CBD-SA) (21). The inventors evaluated engineered CBD-SA as a tumor-targeted drug carrier, leading to improved anti-tumor efficacy by efficient Dox delivery to the tumor microenvironment.

B. Results

1. CBD-SA Binds to Collagen and Can be Conjugated to Dox

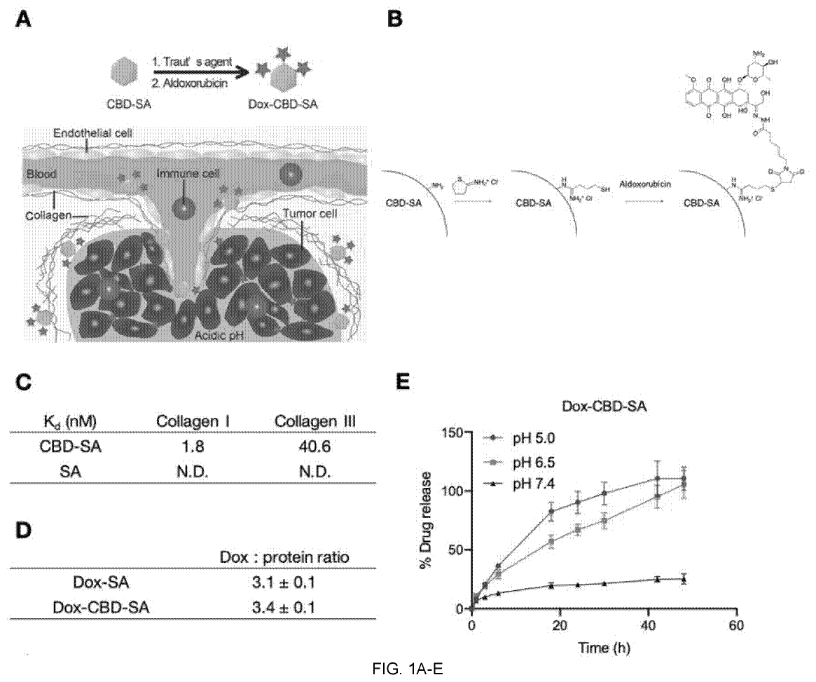

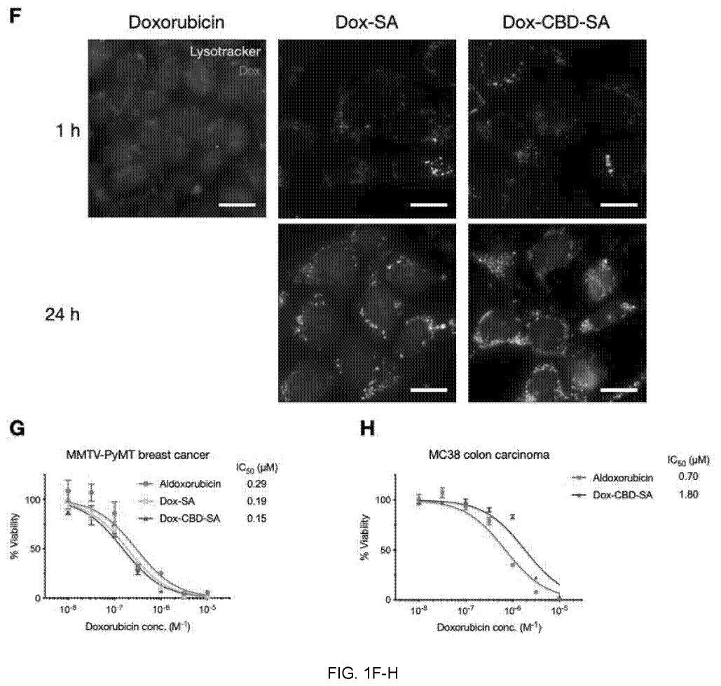

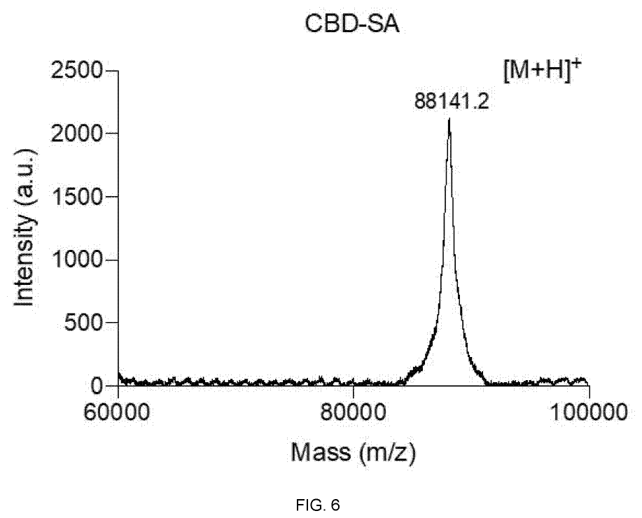

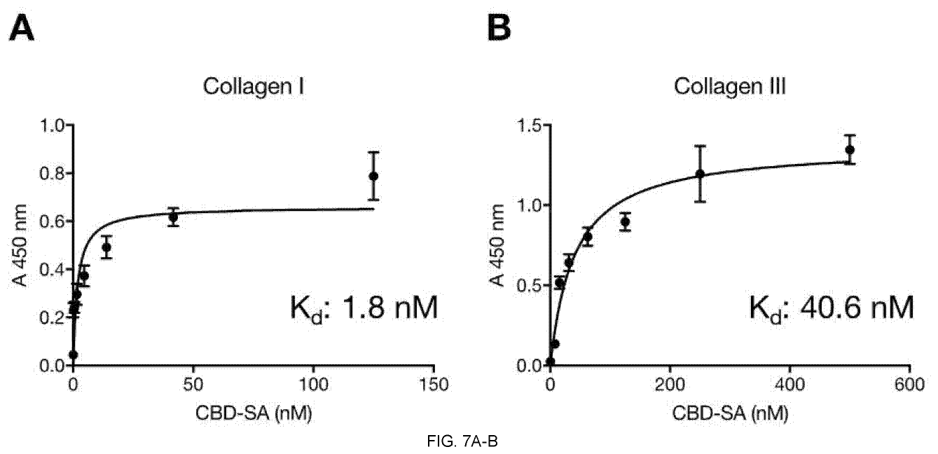

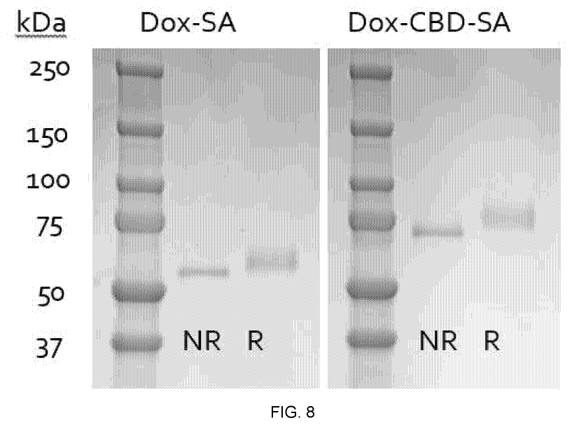

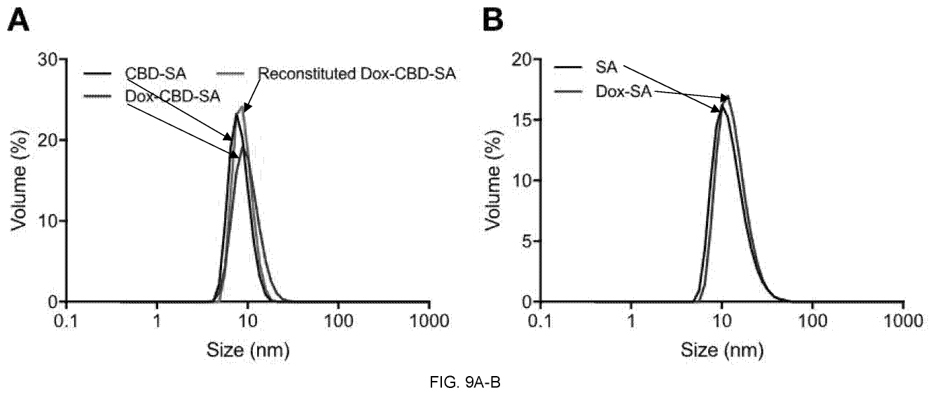

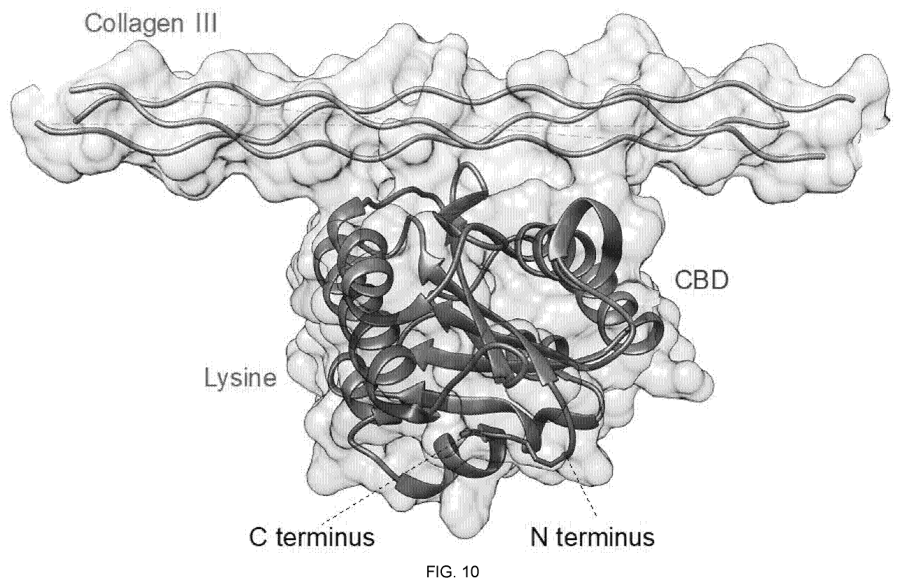

The inventors synthesized Dox-CBD-SA conjugates to target the tumor microenvironment (FIG. 1A, B). The inventors first investigated the binding abilities of CBD-SA to recombinant collagen protein in vitro. SA was expressed recombinantly, with the CBD on the N-terminus of mouse SA using a (GGGS)₂ linker (SEQ ID NO:3). The molecular weight of CBD-SA was analyzed by MALDI-TOF MS (FIG. 6). Strong binding affinities (nM range dissociation constant (Kd) values) of CBD-SA to collagen type I and type III were observed (FIG. 1C, FIG. 7). For Dox conjugation, the inventors first thiolated the lysine residues of CBD-SA using 2-iminothiolane (also known as Traut's reagent). Then, aldoxorubicin was covalently conjugated to CBD-SA. Unmodified SA was also conjugated with aldoxorubicin in the same way (Dox-SA). SDS-polyacrylamide gel electrophoresis (PAGE) under non-reducing condition showed that purified Dox-SA and Dox-CBD-SA are monomeric (FIG. 8). Before and after Dox conjugation, the hydrodynamic size of CBD-SA was measured (FIG. 9). The results also showed that CBD-SA exists in a monomeric form, and Dox conjugation did not alter this character even after a lyophilization/reconstitution cycle. Approximately 3 Dox molecules were conjugated per SA molecule and per CBD-SA molecule (FIG. 1D). Notably, the inventors' conjugation method would not affect the binding ability of CBD-SA to collagens, since there are no cysteine or lysine residues at the binding interface between the VWF A3 domain and human collagen III (FIG. 10, PDB: 4DMU (22)). This interface is also far from the C-terminal fusion site to the SA domain.

2. Dox is Released Under Acidic pH Conditions

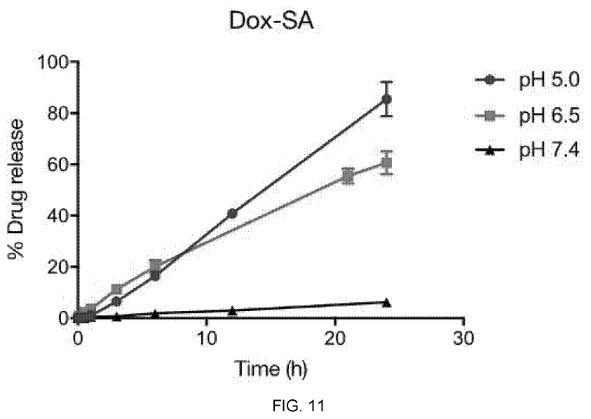

Because Dox is linked to SA with a pH-sensitive cleavable linker, the release kinetics of Dox from conjugates under different pH conditions were examined (FIG. 1E). After 48 h of incubation, Dox release from Dox-CBD-SA reached a maximum at pH 5.0 and pH 6.5 (reported tumor microenvironment condition). In contrast, only about 20% of Dox was released at pH 7.4 after 48 h. Dox-SA showed similar release profiles (FIG. 11). These data show the pH-dependent release of Dox from conjugates, consistent with previously reported release kinetics of small chemicals linked via a hydrazone linkage (21).

3. Dox Conjugates are Taken up by Cancer Cells and Retain Cytotoxicity

The inventors compared the intracellular localization of Dox conjugates with free drug using confocal laser scanning microscopy by detecting the fluorescence of Dox. Because Dox is a major drug for breast cancer (23), here the inventors chose mouse mammary tumor virus-polyomavirus middle T antigen (MMTV-PyMT) murine breast cancer as an experimental model. The MMTV-PyMT cells were cultured in the presence of Dox or Dox conjugates and then their intracellular uptake was assessed (FIG. 1F). After 1 h of incubation, free Dox was detected in cytoplasm, intracellular acidic organelles, and preferentially in the nucleus, indicating that its delivery is mediated by passive transmembrane diffusion. In contrast, 1 h after addition of either Dox-SA or Dox-CBD-SA, the cytoplasm did not show strong fluorescence compared to the unconjugated Dox. Rather, punctuate fluorescence was observed, with some puncta co-localized with lysosomes, suggesting that Dox-SA and Dox-CBD-SA were both internalized via endocytosis. 24 h after the addition of Dox-conjugates, Dox-derived fluorescence was observed in the nucleus as well, suggesting that the acidic pH in intracellular organelles induced drug liberation from the conjugates. The inventors next examined the cytotoxicity of the different Dox forms in vitro. MMTV-PyMT cells or MC38 colon carcinoma cells were seeded and incubated in the presence of the Dox forms for 3 days. Viability tests showed that all three Dox forms have comparable cytotoxicity in vitro (FIG. 1G, H).

4. Dox-CBD-SA Demonstrates Comparable Blood Plasma Pharmacokinetics as Aldoxorubicin and Accumulates in Tumors

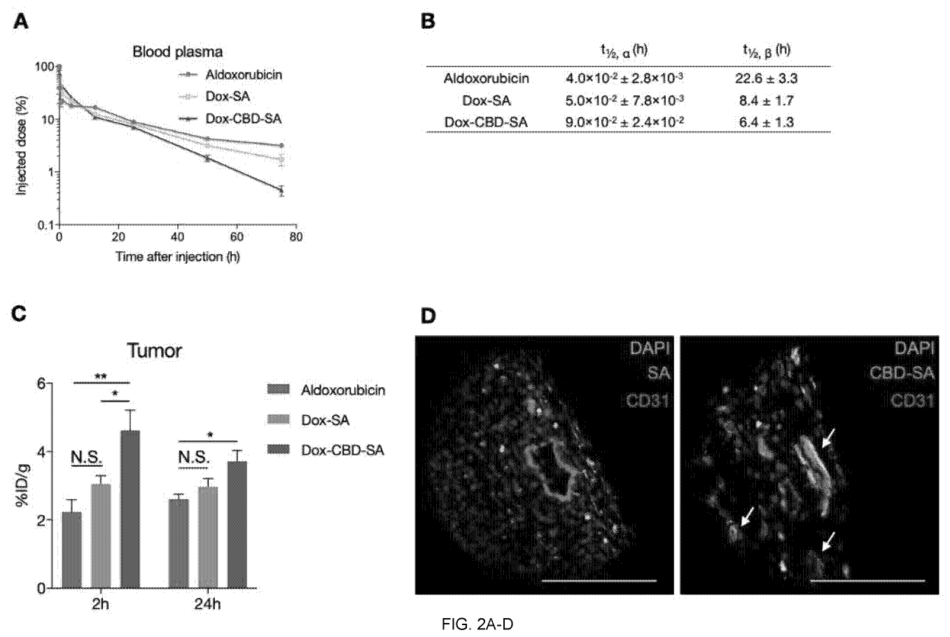

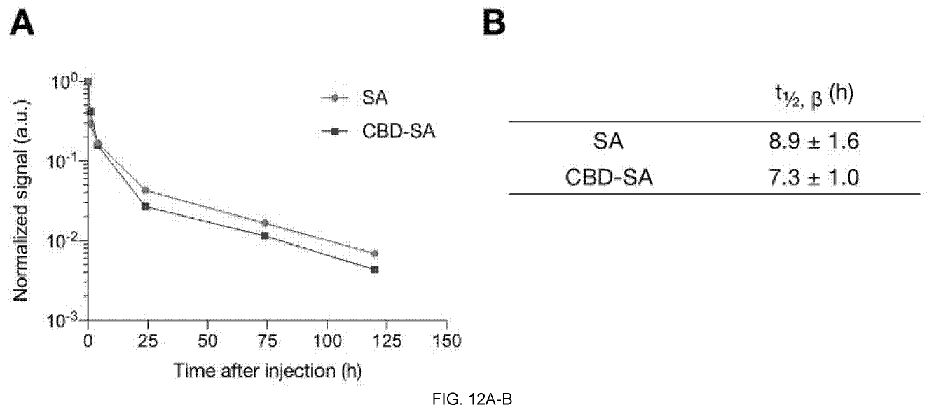

Aldoxorubicin reacts with endogenous SA rapidly after intravenous (i.v.) administration, therefore it possesses substantially longer blood plasma half-life compared with Dox (18). The inventors tested the plasma pharmacokinetics of aldoxorubicin with or without prior conjugation of SA and CBD-SA using tumor-free FVB mice. After i.v. injection, similar blood plasma half-life of aldoxorubicin, Dox-SA, and Dox-CBD-SA were observed (FIG. 2A, B). The inventors also examined the plasma pharmacokinetics of fluorescently labeled SA and CBD-SA with a pH-insensitive linker (FIG. 12). The result showed that the half-lives of each protein conjugated with either Dox or dye were similar, suggesting that Dox liberation from the conjugates does not occur in the blood circulation.

The inventors next hypothesized that CBD-fusion to SA would increase the amount of Dox within the tumor via active targeting against collagens within the tumor microenvironment. To test this hypothesis, the inventors measured the amounts of Dox within tumor tissues after a single i.v. administration. Dox-CBD-SA showed significantly higher tumor accumulation of Dox compared to aldoxorubicin and Dox-SA at 2 h post administration (FIG. 2C). Conjugation with CBD-SA achieved the highest tumor accumulation of Dox after 24 h of injection as well, showing a significant increase compared to aldoxorubicin. Histological analysis revealed that fluorescently-labeled CBD-SA co-localized with CD31 staining within tumor tissue, demonstrating that CBD-SA targets the tumor vasculature (FIG. 2D). These data demonstrate that CBD fusion to SA to which Dox is conjugated enables Dox to target tumors, resulting in enhanced tumor accumulation of Dox.

5. Dox-CBD-SA Demonstrates Superior Efficacy in MMTV-PyMT Murine Breast Cancer Model

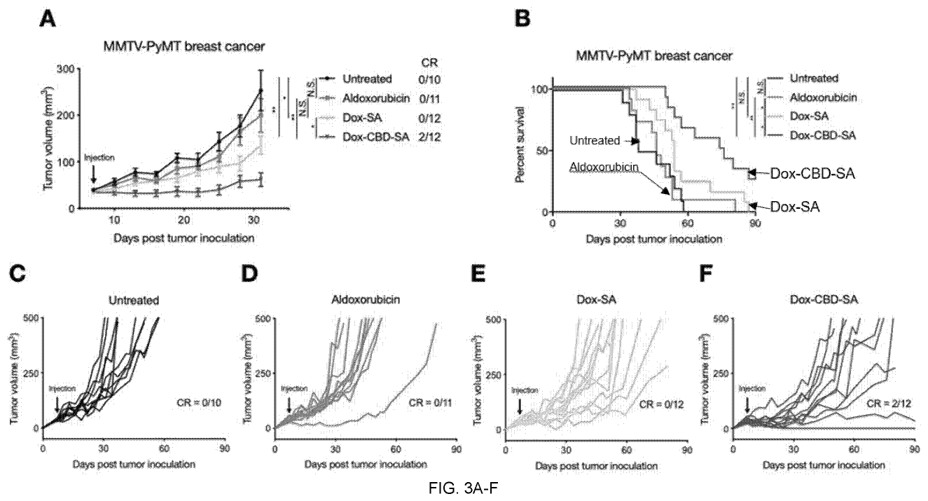

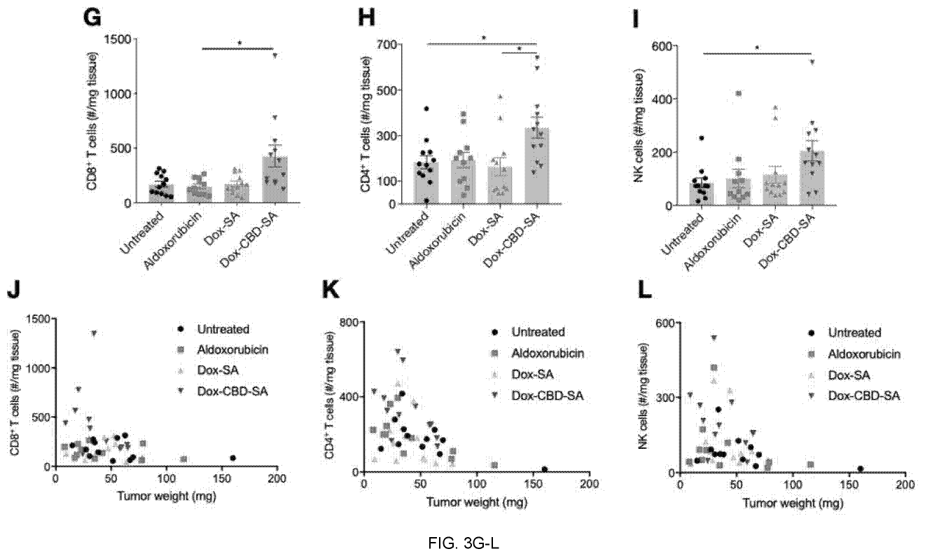

Motivated by the plasma pharmacokinetics and tumor accumulation studies, the inventors evaluated the anti-tumor effects of Dox-CBD-SA in vivo. MMTV-PyMT orthotopic tumor-bearing mice received a single i.v. injection of the Dox forms (5 mg/kg on a Dox basis) via the tail vein. Dox-SA and Dox-CBD-SA significantly suppressed tumor growth, whereas aldoxorubicin did not (FIG. 3A, C-F). This suggests that pre-conjugation of Dox with SA would provide a higher therapeutic effect than in situ conjugation of aldoxorubicin with endogenous SA. Notably, Dox-CBD-SA showed a greater therapeutic effect compared to Dox-SA. Dox-CBD-SA treatment significantly extended the survival rate compared to all the other groups (FIG. 3B) and induced complete tumor remission in 2 mice out of 12. These data demonstrate that CBD-fused SA functions as a superior Dox carrier compared to unmodified SA in terms of anti-tumor efficacy.

6. Dox-CBD-SA Enhances Tumor Infiltration of Lymphocytes

Dox reportedly induces ICD, which stimulates immune responses against antigens from necrotic cells (15). Indeed, ICD increases the number of tumor-infiltrating lymphocytes (TILs), which is a marker of favorable prognosis in multiple types of cancers such as colorectal cancer and breast cancer (24, 25). The inventors analyzed the TILs after Dox-CBD-SA treatment, particularly T cells and natural killer (NK) cells. Lymphocytes were extracted from the tumor and analyzed by flow cytometry 7 days after treatment with the various Dox forms. Dox-CBD-SA, but not aldoxorubicin or Dox-SA, significantly increased the numbers of CD8⁺ T cells, CD4⁺ T cells, and NK cells within the tumor per unit tumor mass (FIG. 3G-I). In particular, Dox-CBD-SA treatment increased the number of CD8⁺ T cells more than two-fold higher than the other treatment groups (FIG. 3G). Plots of individual tumor size and TIL cell number revealed Dox-CBD-SA indeed induced a correlation between small tumor size and the number of tumor-infiltrated CD8⁺ T cells, CD4⁺ T cells and NK cells (FIG. 3J-L). These data suggest that enhanced infiltration of lymphocytes, particularly CD8⁺ cytotoxic T cells, may contribute to the superior anti-tumor effects of Dox-CBD-SA.

7. Dox-CBD-SA Shows Reduced Toxicity

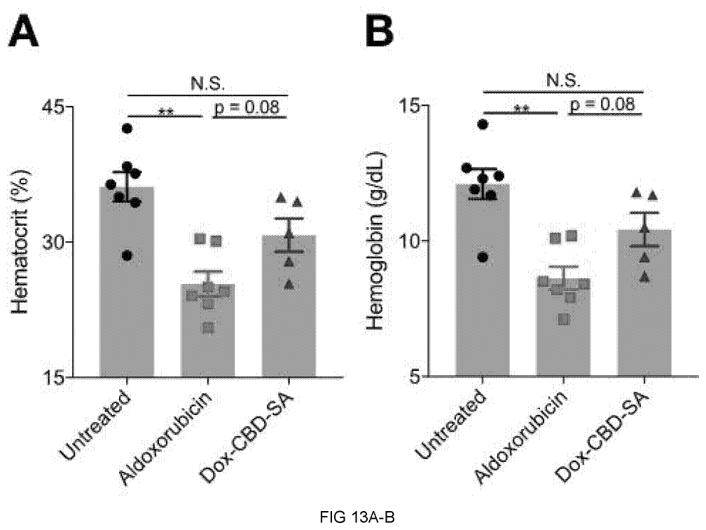

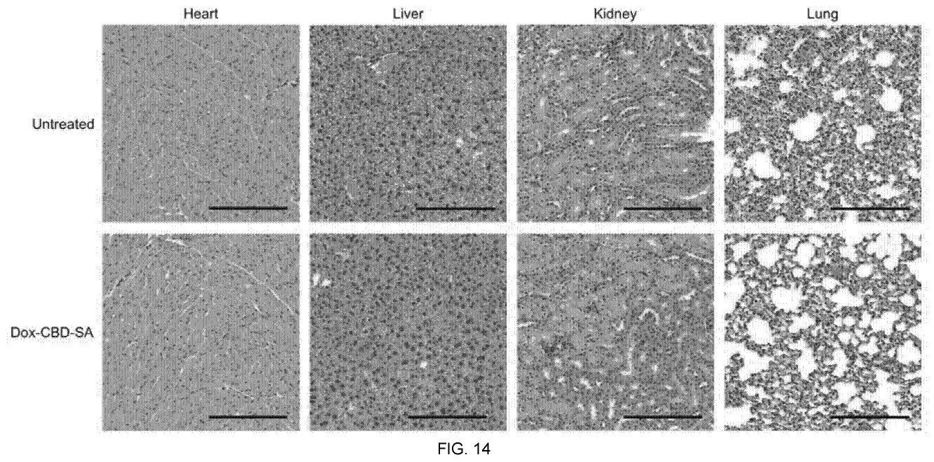

Because conjugated aldoxorubicin is only released very slowly from SA under physiological pH (FIG. 1E), the inventors hypothesized that Dox-CBD-SA shows reduced toxicity compared to aldoxorubicin. The inventors evaluated adverse events after a single injection of aldoxorubicin or Dox-CBD-SA (20 mg/kg on a Dox basis) using tumor-free FVB mice. Administration of aldoxorubicin increased the plasma concentration of inflammatory cytokines such as IFN-γ, TNF-α, IL-5, and IL-6, whereas Dox-CBD-SA did not (FIG. 4A-D). Aldoxorubicin treatment also induced a significant decrease in red blood cell (RBC) counts, white blood cell (WBC) counts, hematocrit, and hemoglobin concentration (FIG. 4E, F, FIG. 13). In contrast, adverse effects of Dox-CBD-SA on hematological values were mild. Only a significant decrease in WBC counts compared to the untreated group was observed. Aldoxorubicin administration induced splenomegaly, whereas Dox-CBD-SA treatment did not (FIG. 4G). Histological analysis revealed that Dox-CBD-SA treatment provided no observable damage in heart, liver, kidney, or lung (FIG. 14). These data suggest that pre-conjugation of Dox with CBD-SA reduced toxicity in various aspects.

8. Dox-CBD-SA in Combination with Anti-PD-1 Antibody (αPD-1) Eradicates MC38 Tumor

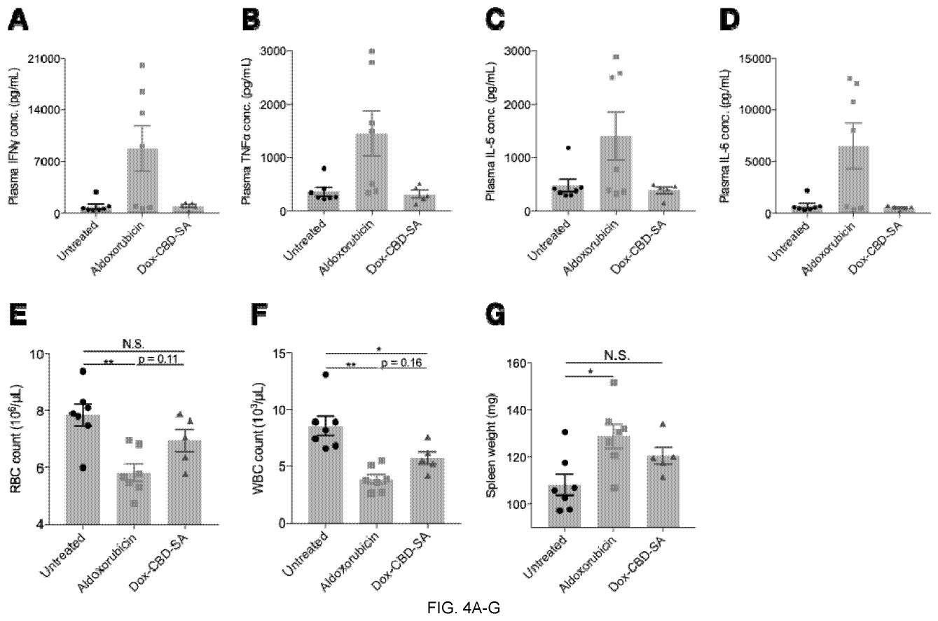

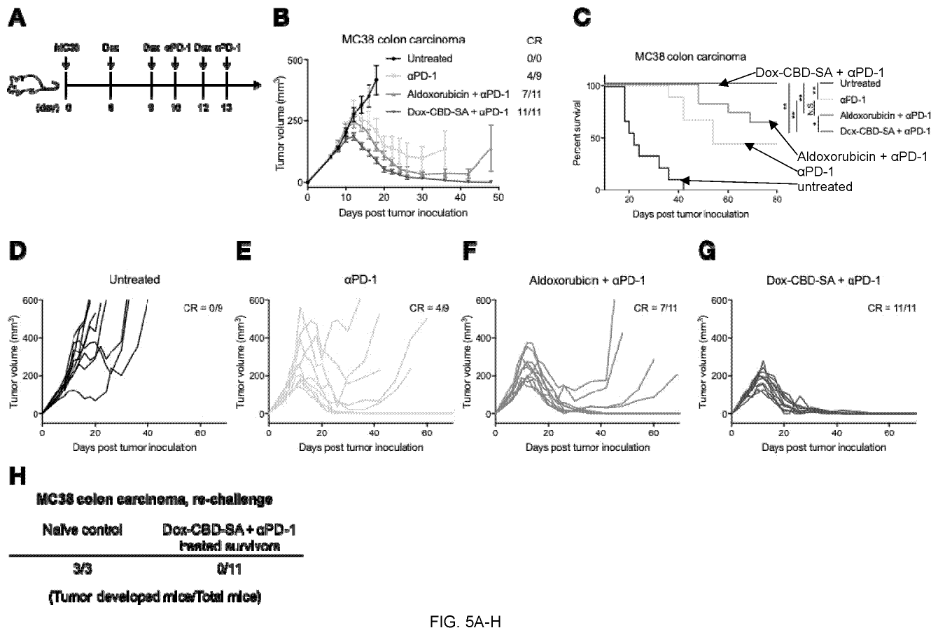

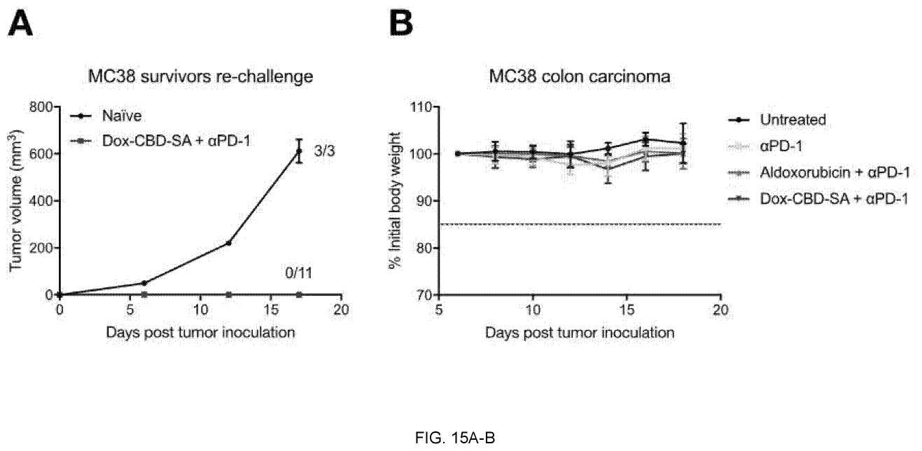

Based on the observation of increased TILs induced by Dox-CBD-SA treatment (FIG. 3G-L), the inventors hypothesized that Dox-CBD-SA combination therapy with CPI would show a greater therapeutic effect compared to aldoxorubicin combination therapy with CPI. To test this hypothesis, the inventors selected αPD-1 as the most widely used CPI in the clinic (26). Importantly, αPD-1 is used in combination with Dox in clinical trials (e.g. NCT02648477). The inventors examined the anti-tumor effect of aldoxorubicin and Dox-CBD-SA in combination with αPD-1 using the MC38 colon carcinoma model, which is immunogenic (27), but not curable by Dox monotherapy (28). C57BL/6 mice were inoculated subcutaneously with 5×10⁵ MC38 cells. The treatment schedule is shown in FIG. 5A. Aldoxorubicin or Dox-CBD-SA was administered to mice 6, 9, and 12 days after tumor inoculation. Since Dox-CBD-SA increases the number of TILs, the inventors injected 100 μg of αPD-1 one day after Dox treatment for two times (on day 10 and day 13). Dox-CBD-SA+αPD-1 therapy completely eradicated established MC38 tumors (average tumor volume was about 100 mm³ on day 6, FIG. 5B, G), and significantly prolonged the survival of mice compared to all the other groups (FIG. 5C). In other treatment groups, a fraction of mice failed to show a complete response, and average tumor size increased gradually (FIG. 5B, D-F). In Dox-CBD-SA+αPD-1 treated survivors, no mice re-challenged with MC38 cells without additional therapy developed palpable tumors, demonstrating that they had acquired strong immunologic anti-tumor memory (FIG. 5H, FIG. 15A). During the treatments, no mouse showed more than 15% body weight loss (FIG. 15B). These data show that Dox-CBD-SA, through induction of ICD, synergizes with αPD-1 to show further anti-tumor effects that could not be achieved by equivalent doses of aldoxorubicin+αPD-1.

C. Discussion

Because small molecule anticancer drugs broadly distribute to tissues and induce systemic side effects, modifications of drugs to improve their pharmacokinetics and bio-distribution have been attempted. Nanoparticle-formulated (17) or SA-reactive (18, 19) doxorubicin exhibits improved pharmacokinetics and accumulation within tumors based in part on their pathologically abnormal vasculature (5). However, this effect may not always be effective in human cancers because of their heterogeneity (29). Thus, drugs that are dependent on passive targeting alone may have room for improvement. Active targeting of tumor-specific or tumor-associated antigens for drug delivery is another therapeutic strategy. However, this intrinsically limits the applicable range of cancers and may also lead acquired drug resistance due to antigen-selective cell targeting and killing, which antigen may be lost by mutation (30). Here, the inventors engineered CBD-SA to overcome these issues. Unlike other active targeting strategies, CBD-SA does not require the prior investigation of tumor-associated antigen expression, because collagen is nearly ubiquitously expressed in tumors, and the CBD gains access to the tumor stroma via the abnormal blood vessel structure within the tumor microenvironment (6). Subsequently, the CBD-SA binds to exposed collagen (FIG. 1C, FIG. 7) and converts the tumor stroma into a reservoir for chemotherapeutics. Dox conjugation to CBD-SA showed significantly higher accumulation of Dox within tumor tissue compared to aldoxorubicin and Dox-SA (FIG. 2C). After accumulation of Dox-CBD-SA within the tumor tissue, the hydrazone linkage, which can be cleaved under the slightly acidic conditions in the tumor microenvironment (FIG. 1E) (21), enables the sustained release of Dox from CBD-SA. At the same time, it is known that tumor cells uptake SA (1). Notably, CBD fusion did not alter the cellular uptake of SA (FIG. 1F), indicating that Dox-CBD-SA can also be delivered intracellularly as efficient as Dox-SA. Thus, part of the Dox release may occur in the tumor stroma while the Dox-CBD-SA is still matrix-bound, and part may occur in the endolysosomal compartment following endocytosis. The relatively low molecular weight of CBD-SA (88 kDa, FIG. 6) may be a benefit in terms of diffusion into tumor tissues (32).

In terms of anti-tumor efficacy, Dox-CBD-SA significantly suppressed the growth of MMTV-PyMT breast cancer and extended the survival of mice compared to aldoxorubicin and Dox-SA (FIG. 3A-F). Because Dox-CBD-SA showed the highest accumulation into tumor tissue in vivo, Dox-CBD-SA should induce tumor cell death more efficiently via inhibition of tumor cell proliferation. In addition to this effect, a single injection of Dox-CBD-SA brought a long-lasting therapeutic effect in spite of its faster plasma clearance half-life (FIG. 2A, B). This could be explained by the inventors' observation that Dox-CBD-SA treatment induces a higher number and density of TILs compared to Dox-SA and aldoxorubicin treatments (FIG. 3G-L). Therefore, the anti-tumor mechanism of action of Dox-CBD-SA may be not only direct cell killing, but also the stimulation of host anti-tumor immunity. Since Dox-CBD-SA efficiently accumulates within tumors, it may induce ICD and tumor antigen exposure to the immune system more efficiently than aldoxorubicin and Dox-SA. As a consequence, Dox-CBD-SA synergistically eradicated MC38 colon carcinoma when administered in combination with αPD-1 (FIG. 5B, G). Improved therapeutic efficacy of Dox-SA and Dox-CBD-SA in comparison with aldoxorubicin (FIG. 3A-F) also indicates that pre-conjugation of Dox before injection would provide higher anti-tumor efficacy. In addition to rapid clearance from blood circulation, in situ conjugation of aldoxorubicin with other sulfhydryl compounds such as cysteine, glutathione, fibronectin, or al-antitrypsin in plasma (18) is also a possible cause of inefficient therapeutic efficacy of aldoxorubicin.

Cardiac toxicity is a major drawback of Dox, which limits the lifetime cumulative dose of Dox (13). Histological analysis revealed that even 20 mg/kg of Dox-CBD-SA administration did not show any signs of cardiac damage (FIG. 14). This suggests that Dox pre-conjugated with CBD-SA is less cardiotoxic than free Dox, which irreversibly damages cardiac tissue at a cumulative dose of 15 mg/kg in mouse models (34). Importantly, a cumulative dose of 15 mg/kg is nearly equivalent to the maximum cumulative dose in human (35).

In terms of the manufacturing process, the inventors conjugated Dox using Traut's reagent, which allows precise control of the drug conjugation ratio (36). This method has little risk to abrogate binding between the CBD and collagen, since there are no lysine residues at the binding interface between the VWF A3 domain and collagen (FIG. 10) (22). Moreover, SA contains approximately 7-fold the number of lysine residues as the CBD sequence, also suggesting the low risk of unfavorable conformational changes in the CBD due to conjugation. Traut's reagent is also used for an ADC targeting CD70 (MDX-1203, Bristol-Myers Squibb) (37), indicating its translational applicability. As CBD-SA is produced with high yield (˜70-100 mg/L of HEK293 cell culture), the inventors propose that pre-conjugation of Dox to CBD-SA produces high anti-tumor efficacy with a simple and translatable production method.

The reduction in non-specific toxicity is unexpected, since one may expect CBD-SA to accumulate in undesirable sites in the body such as liver, kidney, and wounds, where collagens may be exposed via a fenestrated or leaky endothelium. However, the inventors did not observe pathological damage in the liver and kidney after 20 mg/kg of Dox-CBD-SA administration (FIG. 14). Furthermore, the increased efficacy of the current polypeptides is somewhat unexpected, since prior work in this field has shown that chemical conjugation may decrease the half-life of SA in general. Methotrexate conjugation reportedly accelerated the clearance of methotrexate-SA conjugates from circulation in a drug:protein ratio dependent manner (38). Thus, it is surprising that an increase in therapeutic efficiency was achieved even though the half-lives of Dox-SA and Dox-CBD-SA were shorter than the reported half-life of naïve mouse SA (t½, β=35 (h)) (39).

In conclusion, Dox-CBD-SA accumulated into tumors and activated host anti-tumor immunity. As a consequence, monotherapy of Dox-CBD-SA suppressed orthotopic MMTV-PyMT breast tumor growth and prolonged survival. More importantly, combination therapy of Dox-CBD-SA with immune checkpoint inhibition via αPD-1 completely eradicated tumors in the immunogenic MC38 model. CBD fusion provided an active targeting ability to SA, which is classically used as a passively targeted drug carrier, enabling effective drug delivery to tumors from the systemic circulation. CBD-SA is expected to be non-immunogenic and biologically acceptable, because it is comprised of two proteins (VWF A3 domain and SA) that naturally exist in the blood. Furthermore, CBD-SA acts independency of tumor type-specific antigens and thus provides broad applicability to various types of solid tumors as a drug carrier. Therefore, CBD-SA may hold potential for clinical translation to cancer therapy as an anti-tumor drug carrier.

D. Materials & Methods

1. Study Design

This study was designed to verify the strategy for anti-cancer drug delivery to tumors by engineered collagen-binding SA as a drug conjugation carrier. Specifically, the inventors tested if anti-tumor efficacy of Dox-CBD-SA against mouse models of breast cancer and colon carcinoma are improved compared to their unmodified forms. The adverse effects of Dox-CBD-SA were also tested using tumor-free mice. The inventors measured tumor growth, anti-cancer immune responses, and multiple aspects of toxicity after treatment. Statistical methods were not used to predetermine required sample size, but sample sizes were determined based on estimates from pilot experiments and previously published results such that appropriate statistical tests could yield significant results. CBD-SA was produced by multiple individuals to ensure reproducibility. All experiments were replicated at least twice except for FIG. 12 (once). For animal studies, mice were randomized into treatment groups within a cage immediately before the first Dox-CBD-SA injection and treated in the same way. Samples were excluded from analysis only when an animal developed a health problem for a non-treatment related reason, according to the animal care guidelines. The survival endpoint was reached when the tumor size became over 500 mm³ for MMTV-PyMT model, and 600 mm³ for MC38 model. The n values used to calculate statistics are indicated in the figures or in the figure legends. Drug administration and pathological analyses were performed in a blinded fashion. Statistical methods are described in the “Statistical analysis” section.

2. Cell Culture

Mouse mammary tumor virus-polyomavirus middle T antigen (MMTV-PyMT) cells were obtained from spontaneously developed breast cancer in FVB-Tg (MMTV-PyMT) transgenic mice as described previously (9). The MC38 colon carcinoma cell line was kindly provided by the R. Weichselbaum laboratory (University of Chicago). DMEM (Gibco) supplemented with 110 mg/L of sodium pyruvate, 10% heat inactivated FBS, and 1% penicillin/streptomycin was used for both cell lines. The cell lines were checked for mycoplasma contamination by an IMPACT I pathogen test (IDEXX BioResearch).

3. Mice

Female FVB mice, ages 8 to 12 weeks, were obtained from Charles River and Jackson Laboratory. Female C57BL/6 mice, ages 8 to 12 weeks, were obtained from Jackson Laboratory. All the animal experiments performed in this work were approved from the Institutional Animal Care and Use Committee of the University of Chicago.

4. Production and Purification of CBD-SA

CBD-SA protein was designed, produced and purified similarly to previously reported CBD proteins (9). The sequences encoding for the fusion of human VWF A3 domain residues Cys1670-Glyl874 (907-1111 of mature VWF) and mouse SA without pro-peptide (25-608 amino acids of whole SA) were synthesized and subcloned into the mammalian expression vector pcDNA3.1(+) by Genscript. A sequence encoding for a His-tag (6 His) was inserted at the C-terminus for further purification of the recombinant protein. Suspension-adapted HEK-293F cells were routinely maintained in serum-free FreeStyle 293 Expression Medium (Gibco). On the day of transfection, cells were diluted into fresh medium at a density of 1×10⁶ cells/mL. 2 μg/mL plasmid DNA, 2 μg/mL linear 25 kDa polyethylenimine (Polysciences), and OptiPRO SFM media (4% final concentration, Thermo fisher scientific) were added. The culture flask was agitated by orbital shaking at 135 rpm at 37° C. in the presence of 5% CO₂. 7 days after transfection, the cell culture medium was collected by centrifugation and filtered through a 0.22 μm filter. Culture media was loaded into a HisTrap HP 5 mL column (GE Healthcare), using an ÄKTA pure 25 (GE Healthcare). After washing of the column with wash buffer (20 mM imidazole, 20 mM NaH₂PO₄, 0.5 M NaCl, pH 7.4), protein was eluted with a gradient of 500 mM imidazole (in 20 mM NaH₂PO₄, 0.5 M NaCl, pH 7.4). The eluent was further purified with size exclusion chromatography using a HiLoad Superdex 200PG column (GE Healthcare). All purification steps were carried out at 4° C. The protein was verified as >90% pure by SDS-PAGE.

5. MALDI-TOF MS

Purified CBD-SA was analyzed by MALDI-TOF MS (Bruker Ultraflextreme MALDI TOF/TOF) as described previously (9). Bruker flexControl™ was used for data acquisition, and Bruker flexAnalysis™ was used for data processing. First, a saturated solution of α-cyano-4-hydroxycinnamic acid (Sigma-Aldrich) was prepared in 50:50 acetonitrile:1% TFA in water as a solvent. CBD-SA in PBS (5 μL, 0.1 mg/mL) and the matrix solution (25 μL) were mixed, and 1 μL of that mixture was dropped on the MTP 384 ground steel target plate. The drop was dried in a nitrogen gas flow. All samples were analyzed using high mass linear positive mode method with 2500 laser shots at the laser intensity of 75%. The measurements were externally calibrated at three points with a mix of carbonic anhydrase, phosphorylase B, and bovine SA.

6. Binding Affinity Assay

The binding affinity of CBD-SA to collagens were tested as described previously (9). 96 well ELISA plates (Greiner Bio-One) were coated with collagen I or collagen III (10 μg/mL each in PBS) for overnight at 37° C., followed by blocking with 2% BSA in PBS with 0.05% Tween 20 (PBS-T) for 1 h at room temperature. Then, wells were washed with PBS-T and further incubated with CBD-SA at increasing concentrations for 2 h at room temperature. After three washes with PBS-T, wells were incubated for 1 h at room temperature with biotin- conjugated Abs against mouse SA. After washes, bound CBD-SA were detected with tetramethylbenzidine substrate by measurement of the absorbance at 450 nm with subtraction of the absorbance at 570 nm. The apparent Kd values were obtained by nonlinear regression analysis in Prism software (version 7, GraphPad) assuming one-site-specific binding.

7. Synthesis of Dox-Conjugates

Mouse SA or CBD-SA was solubilized in PBS containing 2 mM EDTA. 4 molar equivalents of Traut's reagents solved in PBS containing 2 mM EDTA were added and incubated 1 h at room temperature in the dark. Excess Traut's reagents were removed by Zeba spin desalting column (Thermo fisher scientific). 15 molar equivalents of aldoxorubicin (MedChemExpress) dissolved in 10 mM sodium phosphate buffer (pH 5.9) was added and incubated 1 h at room temperature and overnight at 4° C. in the dark. To quench the reaction, 20 molar equivalents of L-cysteine (Sigma-Aldrich (pharma grade) dissolved in PBS containing 2 mM EDTA) against aldoxorubicin was added. Unreacted Dox precipitates were removed by centrifugation (10000×g, 5 min). Supernatant was further purified by Zeba spin desalting column, followed by ultrafiltration using Amicon-Ultra (Merck, 10K MWCO). Concentration of Dox in the final product was quantitated by absorbance at 495 nm, using molar extinction coefficient of 10650 (L·mol-1·cm⁻¹). The concentration of protein content was measured by Pierce BCA Protein Assay Kit (Thermo fisher scientific) according to manufacturer's instructions.

8. Dynamic Light Scattering (DLS)

The hydrodynamic size of Dox-conjugate in PBS was measured using Zetasizer Nano ZS (Malvern). Conjugates were analyzed immediately after synthesis, or lyophilized and stored at −20° C. until use.

9. Sodium Dodecyl Sulfate Polyacrylamide Gel Electrophoresis (SDS-PAGE)

SDS-PAGE was performed as described in the inventors' previous work (9). A 4-20% gradient gel (Bio-Rad) was used, and 0.5 μg of each Dox-conjugate was loaded with or without reduction with 10 mM DTT. After electrophoresis SimplyBlue SafeStain (Thermo fisher scientific) was used for gel staining according to manufacturer's instruction. ChemiDoc XRS+ system (Bio-Rad) was used for image acquisition.

10. pH Dependent Release of Dox from Conjugates

Slide-A-Lyzer MINI Dialysis Device (Thermo fisher scientific, 10K MWCO) was used to determine the release profile of Dox from conjugates. Dox conjugates were diluted in either PBS (pH 6.5 or 7.4) or 0.1 M acetate buffer (pH 5.0) to final concentration of 100 μM (Dox basis). 150 μL of each sample was loaded into the device and dialyzed with 50 mL of buffer. Dialysis was performed using magnetic stirrer. The temperature of the stage was set to 37° C. and samples were protected from light during dialysis. Dialysate was collected at various time-points and stored at 4° C. until the sample collection was finished. Dialysate was loaded onto a 96 well black plate in duplicate (90 μL/well). Fluorescence was determined using excitation at 495 nm and emission at 590 nm. Serial dilution of doxorubicin hydrochloride was prepared in the same buffer to create standard curve.

11. Cellular Uptake of Dox-Conjugates

To evaluate the cellular uptake of Dox-conjugates, MMTV-PyMT cells were seeded in 96 well high content imaging plate (Corning) at 5000 cells/well and incubated overnight. Cells were washed with PBS, and treated with free Dox, Dox-SA, or Dox-CBD-SA dissolved in DMEM (110 mg/L of sodium pyruvate, 10% heat inactivated FBS, 1% penicillin/streptomycin, Phenol red (-)) at the concentration of 50 μM equivalent of Dox. After the incubation, cells were washed twice, treated with 75 nM of Lysotracker Deep Red and further incubated 30 min at 37° C. Cells were washed twice and observed by IX83 microscope (Olympus) with ×60 magnification. Images were processed using ImageJ software (NIH). Scale bar; 20 μm.

12. In Vitro Cytotoxicity

MMTV-PyMT cells or MC38 cells were seeded in a 96 well tissue culture plate (BD Falcon) at 3000 cells/well and incubated overnight. Cells were washed with DMEM (110 mg/L of sodium pyruvate, 10% heat inactivated FBS, 1% penicillin/streptomycin, Phenol red (-)), and 80 μL/well of DMEM was added. Then, serial dilutions of aldoxorubicin, Dox-SA, or Dox-CBD-SA in PBS was added (20 μL/well). Cells were incubated 3 days at 37° C., and the viability was determined using CellTiter 96® AQueous One Solution Cell Proliferation Assay Kit (Promega) according to manufacturer's instructions. Cells treated with 80 μL/well of DMEM +20 μL/well of PBS were defined as 100% viability, whereas the cell-free wells with the same mixture were defined as 0% viability. Half maximal inhibitory concentration (IC₅₀) values were obtained by nonlinear regression analysis in Prism software ([inhibitor] vs. normalized response).

13. Plasma Pharmacokinetics of Dox Conjugates

A previous report about polypeptide-Dox nanoparticles was referred (40) . To measure pharmacokinetics of Dox, 5 mg/kg Dox equivalent of aldoxorubicin, Dox-SA, or Dox-CBD-SA was injected intravenously into female FVB mice. Blood samples were collected in EDTA coated tubes at 5 min, 30 min, 1 h, 4 h, 12 h, 25 h, 50 h, and 75 h after injection. Blood samples were stored at 4° C. until the end of sample collection. The samples were centrifuged (2000×g, 5 min) and plasma was collected. Plasma samples diluted in acidified isopropanol (75 mM HCl, 10% water, 90% isopropanol) were loaded onto a 96 well black plate (100 μL/well). Fluorescence was measured as described above. Plasma samples were also collected from mice which received no injections, diluted in acidified isopropanol, and measured to create the standard curve of background fluorescence. Exponential two-phase decay (Y=Ae^{-αt}+Be^{-αt}) fitting was used to calculate the plasma half-life. Fast clearance half-life: t_{1/2}, □, slow clearance half-life: t_{1/2}, α. Data was analyzed using Prism software (v7, GraphPad).

14. Plasma Pharmacokinetics of SA and CBD-SA

SA and CBD-SA was labeled with DyLight 800 NHS ester (Thermo fisher scientific) according to the manufacturer's instructions. Unreacted dye was removed by Zeba spin desalting column as described above. After labeling, 200 μg of each protein was injected intravenously into female FVB mice. Blood samples were collected in EDTA coated tubes at 1 min, 1 h, 4 h, 24 h, 74 h, and 120 h after injection. Blood samples were stored at 4° C. until the end of sample collection. The samples were centrifuged (2000×g, 5 min) and plasma was collected. Plasma samples were diluted in PBS and loaded into a 96 well black plate (100 μL/well). The concentration of each protein in plasma was measured with a LI-COR Infrared Odyssey Imager (Li-COR Biosciences). The method of curve fitting and calculation of plasma half-life was described above.

15. MMTV-PyMT Tumor Inoculation and Treatments

The MMTV-PyMT murine breast cancer model was prepared as described previously (9). A total of 5×10⁵ MMTV-PyMT cells suspended in 50 μL of PBS were injected subcutaneously into the mammary gland on the right side of each mouse. Mice were treated with aldoxorubicin, Dox-SA or Dox-CBD-SA on day 7 (5 mg/kg) via tail vein injection. Tumors were measured with a digital caliper at indicated time points, and volumes were calculated as ellipsoids, where V=4/3×3.14×depth/2×width/2×height/2. Mice were sacrificed when tumor volume had reached over 500 mm³ or when active ulceration was observed. For the therapeutic experiment, FVB mice originated from Charles River origin were used. For the tumor infiltrating lymphocytes (TILs) analysis, FVB mice originated from both Jackson Laboratory and Charles River were used. The proportion of mice from different providers was equalized among all groups.

16. MC38 Tumor Inoculation and Treatments

The MC38 murine colon carcinoma model was prepared similarly to Bl6F10 melanoma model as described previously (9). A total of 5×10⁵ MC38 cells suspended in 50 μL of PBS were injected intradermally on the left side of the back of each C57BL/6 mouse. Mice were injected i.v. on day 6, 9, 12 with aldoxorubicin, Dox-SA or Dox-CBD-SA (5 mg/kg). Mice were also treated i.p. with 100 μg of anti-PD-1 (Clone 29F.1A12, Bio X Cell) on day 10 and 13. Tumor growth was monitored as described above. Mice were sacrificed when tumor volume had reached over 600 mm³ or when active ulceration was observed. On day 60, naïve C57BL/6 mice or tumor-free survivors were re-challenged by intradermal injection of 5×10⁵ MC38 cells.

17. Tumor Accumulation Study

Previous report about polypeptide-Dox nanoparticles was referred (40). Aldoxorubicin, Dox-SA, or Dox-CBD-SA was injected to FVB mice with established tumor at 4.16 mg/kg via tail vein. Tumor was collected, weighed, and put on ice 2 h or 24 h after injection. Tumor tissues were suspended in 1 mL of acidified isopropanol, and homogenized using Lysing Matrix D and FastPrep-24 5 G (MP Biomedical) for 40 s at 5000 beats/min. After homogenization, samples were protected from light, and incubated overnight at 4° C. Samples were centrifuged (5000×g, 5 min), and the supernatants were loaded onto a 96 well black plate (100 μL/well, triplicate). Fluorescence was measured to quantify the amount of Dox in tissue extracts as described above. Tumors from untreated mice were also processed, and serial dilutions of tissue extracts were measured to obtain the standard curves of tissue derived auto-fluorescence.

18. Histological Analysis of Injected CBD-SA within Tumor

Mouse SA (Sigma-Aldrich) and CBD-SA were conjugated with NHS-DyLight 488 according to manufacturer's instructions. Unreacted dye was removed by Zeba spin desalting column, then fluorescent protein solution was stored at 4° C. until use. 100 μg of fluorescent labeled SA or CBD-SA labeled with equimolar of dye was intravenously injected to MMTV-PyMT tumor-bearing mice. 1 h after injection, tumors were harvested and frozen in dry ice with OCT compound. 10 μm of tissue slices were obtained by cryo-sectioning. The tissues were fixed with 2% paraformaldehyde in PBS for 15 min at room temperature. After wash with PBS-T, the tissues were blocked with 2% BSA in PBS-T for 1 h at room temperature. The tissues were stained with biotin labeled anti-mouse CD31 antibody (1:100, Biolegend) and Alexa Fluor 647 streptavidin (1:1000, Biolegend). The tissues were washed three times, then covered with ProLong gold antifade mountant with DAPI (Thermo fisher scientific). IX83 microscope (Olympus) was used for imaging with ×60 magnification. Images were processed using ImageJ software (NIH).

19. Flow Cytometry and Antibodies

MMTV-PyMT model was prepared as described above. Mice were treated on day 7 with aldoxorubicin, Dox-SA or Dox-CBD-SA (5 mg/kg). Mice were sacrificed on day 14. Cell suspensions were obtained from each tumor as described previously (9). Tumors were harvested and digested in Dulbecco's Modified Eagle Medium (DMEM) supplemented with 2% FBS, 2 mg/mL collagenase D and 40 μg/mL DNase I (Roche) for 30 min at 37° C. Single-cell suspensions were obtained by gently disrupting the organs through a 70 μm cell strainer. Red blood cells were lysed with ACK lysing buffer (Quality Biological). Fixable live/dead cell discrimination was performed using Fixable Viability Dye eFluor 455 (eBioscience) according to the manufacturer's instructions. Following a washing step, cells were stained with specific antibodies for 20 min on ice prior to fixation. Following antibodies were used to stain the cells: CD3 (145-2C11, BD Biosciences), CD4 (RM4-5, BD Biosciences), CD8α (53-6.7, BD Biosciences), CD45 (30-F11, BD Biosciences), and NK1.1 (PK136, BD Biosciences). All flow cytometric analyses were done using a Fortessa flow cytometer (BD Biosciences) and analyzed using FlowJo software (Tree Star).

20. Toxicity Profiles

Tumor-free FVB mice received 20 mg/kg of aldoxorubicin or Dox-CBD-SA by intravenous injection. Blood samples were collected from each mouse in EDTA-coated tube by submandibular bleeding on day 3 and day 6 after injection for plasma cytokine analysis and hematological analysis. Body weight of each mouse was measured at indicated time points. On day 16, mice were sacrificed and organs were harvested. Spleens were weighed, and the other organs were used for histological analysis. Mice were sacrificed when more than 15% decrease of initial body weight was observed.

21. Hematological Analysis

Blood samples were analyzed using COULTER Ac⋅T 5diff CP hematology analyzer (Beckman coulter) according to the manufacturer's instructions.

22. Measurement of Plasma Cytokines

Blood plasma was collected from whole blood sample as described above and stored at −20° C. until use. Cytokine concentrations in plasma were measured using Ready-SET-Go! ELISA kits (eBioscience) and Can Get Signal solution (TOYOBO) according to manufacturer's instructions.

23. Histological Analysis of Heart, Liver, Kidney, and Lung

Organs were fixed with 2% paraformaldehyde in PBS overnight. After embedding in paraffin, blocks were cut into 5 μm sections, followed by H&E staining.

24. Statistical Analysis

Statistically significant differences between experimental groups were determined using Prism software (v7, GraphPad) as described previously (9). Where one-way ANOVA followed by Tukey's HSD post hoc test was used, variance between groups was found to be similar by Brown-Forsythe test. For non-parametric data (FIG. 3G), Kruskal-Wallis test followed by Dunn's multiple comparison test was used. Survival curves were analyzed by using the log-rank (Mantel—Cox) test. The symbols * and ** indicate P values less than 0.05 and 0.01, respectively; N. S., not significant.

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

### REFERENCES

The following references and the publications referred to throughout the specification, to the extent that they provide exemplary procedural or other details supplementary to those set forth herein, are specifically incorporated herein by reference.

1. F. Kratz, Albumin as a drug carrier: design of prodrugs, drug conjugates and nanoparticles. J. Control. Release 132, 171-183 (2008).

2. J. Morales, Defining the role of insulin detemir in basal insulin therapy. Drugs 67, 2557-2584 (2007).

3. R. Flisiak, I. Flisiak, Albinterferon-alfa 2b: a new treatment option for hepatitis C. Expert. Opin. Biol. Ther. 10, 1509-1515 (2010).

4. M. Green, G. Manikhas, S. Orlov, B. Afanasyev, A. Makhson, P. Bhar, M. Hawkins, Abraxane®, a novel Cremophor®-free, albumin-bound particle form of paclitaxel for the treatment of advanced non-small-cell lung cancer. Ann. Oncol. 17, 1263-1268 (2006).

5. Y. Matsumura, H. Maeda, A new concept for macromolecular therapeutics in cancer chemotherapy: mechanism of tumoritropic accumulation of proteins and the antitumor agent smancs. Cancer Res. 46, 6387-6392 (1986).

6. F. Danhier, 0. Feron, V. Préat, To exploit the tumor microenvironment: passive and active tumor targeting of nanocarriers for anti-cancer drug delivery. J. Control. Release 148, 135-146 (2010).

7. K. Temming, D. L. Meyer, R. Zabinski, E. C. Dijkers, K. Poelstra, G. Molema, R. J. Kok, Evaluation of RGD-targeted albumin carriers for specific delivery of auristatin E to tumor blood vessels. Bioconjug. Chem. 17, 1385-1394 (2006).

8. J. Prakash, L. Beljaars, A. K. Harapanahalli, M. Zeinstra-Smith, A. de Jager-Krikken, M. Hessing, H. Steen, K. Poelstra, Tumor-targeted intracellular delivery of anticancer drugs through the mannose-6-phosphate/insulin-like growth factor II receptor. Int. J. Cancer 126, 1966-1981 (2010).

9. J. Ishihara, A. Ishihara, K. Sasaki, S. S.-Y. Lee, M. Yasui, H. Abe, L. Potin, P. Hosseinchi, K. Fukunaga, M. M. Raczy, L. T. Gray, J.-M. Williford, M. Fukayama, S. J. Kron, M. A. Swartz, J. A. Hubbell, Targeted antibody and cytokine cancer immunotherapies through collagen affinity. Sci. Transl. Med. in press.

10. C. Addi, F. Murschel, G. De Crescenzo, Design and use of chimeric proteins containing a collagen-binding domain for wound healing and bone regeneration. Tissue Eng. Part B. Rev. 23, 163-182 (2017).

11. P. P. Provenzano, D. R. Inman, K. W. Eliceiri, J. G. Knittel, L. Yan, C. T. Rueden, J. G. White, P. J. Keely, Collagen density promotes mammary tumor initiation and progression. BMC Med. 6, 11 (2008).

12. Z.-H. Zhou, C.-D. Ji, H.-L. Xiao, H.-B. Zhao, Y.-H. Cui, X.-W. Bian, Reorganized Collagen in the Tumor Microenvironment of Gastric Cancer and Its Association with Prognosis. J. Cancer 8, 1466-1476 (2017).

13. R. C. Young, R. F. Ozols, C. E. Myers, The anthracycline antineoplastic drugs. N. Engl. J. Med. 305, 139-153 (1981).

14. L. Wang, Q. Chen, H. Qi, C. Wang, C. Wang, J. Zhang, L. Dong, Doxorubicin-induced systemic inflammation is driven by upregulation of toll-like receptor TLR4 and endotoxin leakage. Cancer Res. 76, 6631-6642 (2016).

15. G. Kroemer, L. Galluzzi, O. Kepp, L. Zitvogel, Immunogenic cell death in cancer therapy. Annu. Rev. Immunol. 31, 51-72 (2013).

16. J. Rios-Doria, N. Durham, L. Wetzel, R. Rothstein, J. Chesebrough, N. Holoweckyj, W. Zhao, C. C. Leow, R. Hollingsworth, Doxil synergizes with cancer immunotherapies to enhance antitumor responses in syngeneic mouse models. Neoplasia 17, 661-670 (2015).

17. M. E. O'Brien, N. Wigler, M. Inbar, R. Rosso, E. Grischke, A. Santoro, R. Catane, D. Kieback, P. Tomczak, S. Ackland, Reduced cardiotoxicity and comparable efficacy in a phase III trial of pegylated liposomal doxorubicin HCl (CAELYX™/Doxil®) versus conventional doxorubicin for first-line treatment of metastatic breast cancer. Ann. Oncol. 15, 440-449 (2004).

18. F. Kratz, A. Warnecke, K. Scheuermann, C. Stockmar, J. Schwab, P. Lazar, P. Drückes, N. Esser, J. Drevs, D. Rognan, Probing the cysteine-34 position of endogenous serum albumin with thiol-binding doxorubicin derivatives. Improved efficacy of an acid-sensitive doxorubicin derivative with specific albumin-binding properties compared to that of the parent compound. J. Med. Chem. 45, 5523-5533 (2002).

19. R. Graeser, N. Esser, H. Unger, I. Fichtner, A. Zhu, C. Unger, F. Kratz, INNO-206, the (6-maleimidocaproyl hydrazone derivative of doxorubicin), shows superior antitumor efficacy compared to doxorubicin in different tumor xenograft models and in an orthotopic pancreas carcinoma model. Invest. New Drugs 28, 14-19 (2010).

20. S. P. Chawla, Z. Papai, G. Mukhametshina, K. Sankhala, L. Vasylyev, A. Fedenko, K. Khamly, K. Ganjoo, R. Nagarkar, S. Wieland, First-line aldoxorubicin vs doxorubicin in metastatic or locally advanced unresectable soft-tissue sarcoma: a phase 2b randomized clinical trial. JAMA Oncol. 1, 1272-1280 (2015).

21. M. Prabaharan, J. J. Grailer, S. Pilla, D. A. Steeber, S. Gong, Amphiphilic multi-arm-block copolymer conjugated with doxorubicin via pH-sensitive hydrazone bond for tumor-targeted drug delivery. Biomaterials 30, 5757-5766 (2009).

22. T. H. C. Brondijk, D. Bihan, R. W. Farndale, E. G. Huizinga, Implications for collagen I chain registry from the structure of the collagen von Willebrand factor A3 domain complex. Proc. Natl. Acad. Sci. U.S.A. 109, 5253-5258 (2012).

23. H. M. Kuerer, L. A. Newman, T. L. Smith, F. C. Ames, K. K. Hunt, K. Dhingra, R. L. Theriault, G. Singh, S. M. Binkley, N. Sneige, Clinical course of breast cancer patients with complete pathologic primary tumor and axillary lymph node response to doxorubicin-based neoadjuvant chemotherapy. J. Clin. Oncol. 17, 460-469 (1999).

24. K. M. Ropponen, M. J. Eskelinen, P. K. Lipponen, E. Alhava, V. M. Kosma, Prognostic value of tumour-infiltrating lymphocytes (TILs) in colorectal cancer. J. Pathol. 182, 318-324 (1997).

25. S. Loi, N. Sirtaine, F. Piette, R. Salgado, G. Viale, F. Van Eenoo, G. Rouas, P. Francis, J. P. Crown, E. Hitre, Prognostic and predictive value of tumor-infiltrating lymphocytes in a phase III randomized adjuvant breast cancer trial in node-positive breast cancer comparing the addition of docetaxel to doxorubicin with doxorubicin-based chemotherapy: BIG 02-98. J. Clin. Oncol. 31, 860-867 (2013).

26. K. M. Hargadon, C. E. Johnson, C. J. Williams, Immune checkpoint blockade therapy for cancer: an overview of FDA-approved immune checkpoint inhibitors. Int. Immunopharmacol. 62, 29-39 (2018).

27. M. Yadav, S. Jhunjhunwala, Q. T. Phung, P. Lupardus, J. Tanguay, S. Bumbaca, C. Franci, T. K. Cheung, J. Fritsche, T. Weinschenk, Predicting immunogenic tumour mutations by combining mass spectrometry and exome sequencing. Nature 515, 572-576 (2014).

28. R. Kuai, W. Yuan, S. Son, J. Nam, Y. Xu, Y. Fan, A. Schwendeman, J. J. Moon, Elimination of established tumors with nanodisc-based combination chemoimmunotherapy. Sci. Adv. 4, eaao1736 (2018).

29. U. Prabhakar, H. Maeda, R. K. Jain, E. M. Sevick-Muraca, W. Zamboni, O. C. Farokhzad, S. T. Barry, A. Gabizon, P. Grodzinski, D. C. Blakey, Challenges and key considerations of the enhanced permeability and retention effect for nanomedicine drug delivery in oncology. Cancer Res. 73, 2412-2417 (2013).

30. C. Holohan, S. Van Schaeybroeck, D. B. Longley, P. G. Johnston, Cancer drug resistance: an evolving paradigm. Nat. Rev. Cancer 13, 714-726 (2013).

31. M. Yasunaga, S. Manabe, D. Tarin, Y. Matsumura, Cancer-stroma targeting therapy by cytotoxic immunoconjugate bound to the collagen 4 network in the tumor tissue. Bioconjug. Chem. 22, 1776-1783 (2011).

32. A. Pluen, Y. Boucher, S. Ramanujan, T. D. McKee, T. Gohongi, E. di Tomaso, E. B. Brown, Y. Izumi, R. B. Campbell, D. A. Berk, Role of tumor—host interactions in interstitial diffusion of macromolecules: cranial vs. subcutaneous tumors. Proc. Natl. Acad. Sci. U.S.A. 98, 4628-4633 (2001).

33. N. Diamantis, U. Banerji, Antibody-drug conjugates—an emerging class of cancer treatment. Br. J. Cancer 114, 362-367 (2016).

34. P. W. Fisher, F. Salloum, A. Das, H. Hyder, R. C. Kukreja, Phosphodiesterase-5 inhibition with sildenafil attenuates cardiomyocyte apoptosis and left ventricular dysfunction in a chronic model of doxorubicin cardiotoxicity. Circulation 111, 1601-1610 (2005).

35. G. Takemura, H. Fujiwara, Doxorubicin-induced cardiomyopathy: from the cardiotoxic mechanisms to management. Prog. Cardiovasc. Dis. 49, 330-352 (2007).

36. M. J. McCall, H. Diril, C. F. Meares, Simplified method for conjugating macrocyclic bifunctional chelating agents to antibodies via 2-iminothiolane. Bioconjug. Chem. 1, 222-226 (1990).

37. T. K. Owonikoko, A. Hussain, W. M. Stadler, D. C. Smith, H. Kluger, A. M. Molina, P. Gulati, A. Shah, C. M. Ahlers, P. M. Cardarelli, First-in-human multicenter phase I study of BMS-936561 (MDX-1203), an antibody-drug conjugate targeting CD70. Cancer Chemother. Pharmacol. 77, 155-162 (2016).

38. G. Stehle, H. Sinn, A. Wunder, H. H. Schrenk, S. Schütt, W. Maier-Borst, D. L. Heene, The loading rate determines tumor targeting properties of methotrexate-albumin conjugates in rats. Anticancer. drugs 8, 677-685 (1997).

39. C. Chaudhury, S. Mehnaz, J. M. Robinson, W. L. Hayton, D. K. Pearl, D. C. Roopenian, C. L. Anderson, The major histocompatibility complex-related Fc receptor for IgG (FcRn) binds albumin and prolongs its lifespan. J. Exp. Med. 197, 315-322 (2003).

40. J. A. MacKay, M. Chen, J. R. McDaniel, W. Liu, A. J. Simnick, A. Chilkoti, Self-assembling chimeric polypeptide-doxorubicin conjugate nanoparticles that abolish tumours after a single injection. Nat. Mater. 8, 993-999 (2009).

1. A polypeptide comprising an albumin polypeptide or IgG Fc domain polypetide operatively linked to a collagen binding domain.

2. The polypeptide of claim 1, wherein the polypeptide is operatively linked to a cytotoxic agent.

3. The polypeptide of claim 2, wherein the polypeptide is covalently linked to the cytotoxic agent.

4. The polypeptide of claim 2 or 3, wherein the polypeptide is linked to the cytotoxic agent through a cleavable linker.

5. The polypeptide of claim 4, wherein the cleavable linker comprises a pH-cleavable linker.

6. The polypeptide of claim 5, wherein the linker comprises a hydrazone linker.

7. The polypeptide of claim 5 or 6, wherein the linker is cleaved at a pH of less than 7.4.

8. The polypeptide of any one of claims 1-7, wherein the polypeptide is linked to the cytotoxic agent and/or the collagen binding polypeptide through a bifunctional linker.

9. The polypeptide of any one of claims 2-8, wherein the cytotoxic agent comprises doxorubicin.

10. The polypeptide of any one of claims 1-9, wherein the polypeptide is covalently linked to the collagen binding domain through a peptide bond.

11. The polypeptide of any one of claims 1-10, wherein the polypeptide comprises a collagen binding domain from decorin or von Willebrand factor (VWF).

12. The polypeptide of any one of claims 1-11, wherein the collagen binding domain is at the amino end of the albumin polypeptide or IgG Fc domain polypetide.

13. The polypeptide of any one of claims 1-12, wherein the polypeptide comprises a linker between the albumin polypeptide or IgG Fc domain polypetide and the collagen binding domain.

14. The polypeptide of claim 13, wherein the linker comprises glycine and serine amino acid residues.

15. The polypeptide of claim 14, werien the linker comprises GGGS (SEQ ID NO: 19), (GGGS)ₙ (SEQ ID NO: 20), or (GGGS)₂ (SEQ ID NO: 5).

16. The polypeptide of any one of claims 1-15, wherein the polypeptide is not operatively linked to a particle, nanovesicle, or liposome.

17. The polypeptide of any one of claims 1-16, wherein the polypeptide comprises at least two collagen binding domains.

18. The polypeptide of any one of claims 2-17, wherein the ratio of cytotoxic agent to albumin is 3:1.

19. A composition comprising the polypeptide of any one of claims 1-18.

20. The composition of claim 19, wherein the composition does not comprise a liposome, particle, or nanovescicle.

21. A nucleic acid encoding for the polypeptide of any one of claims 1-18.

22. A cell comprising the nucleic acid of claim 21.

23. A method for making a polypeptide comprising expressing the nucleic acid of claim 21 in a cell and isolated the expressed polypeptide.

24. A method for treating cancer comprising administering the polypeptide of any one of claims 1-18 or the composition of claims 19 or 20.

25. A method for reducing non-specific toxicity of a treatment comprising a cytotoxic agent in a subject, the method comprising administering the polypeptide of any one of claims 2-18 or the composition of claim 19 or 20 to the subject.

26. The method of claim 25, wherein the subject has cancer.

27. The method of claim 25 or 26, wherein the non-specific toxicity is reduced compared to the toxicity of the same cytoxic agent linked to albumin or IgG Fc domain polypetide and unlinked to collagen binding domain.

28. A method for increasing the accumulation of a cytotoxic agent in a tumor in a subject, the method comprising administering the polypeptide of any one of claims 2-18 or the composition of claim 19 or 20 to the subject.

29. The method of claim 28, wherein the accumulation of the cytotoxic agent in the tumor is increased compared to the dose of the same cytoxic agent linked to albumin or IgG Fc domain polypetide and unlinked to collagen binding domain.

30. A method for targeted delivery of a cytotoxic agent to the tumor vasculature, the method comprising administering the polypeptide of any one of claims 2-18 or the composition of claim 19 or 20 to the subject.

31. The method of any one of claim 24, or 26-30, wherein the cancer or tumor comprises a solid tumor.

32. The method of any one of claim 24 or 26-30, wherein the cancer comprises breast or colon cancer or wherein the tumor comprises tumor in the breast or colon.

33. The method of any one of claims 24-32, wherein the method further comprises administration of one or more additional cancer therapies.

34. The method of any one of claims 24-33, wherein the subject has or will receive an immunotherapy.

35. The method of any one of claims 24-34, wherein the method further comprises administration of an immunotherapy.

36. The method of claim 35, wherein the immunotherapy is administered before, after, or concurrent with the polypeptide.

37. The method of any one of claims 34-36, wherein the immunotherapy comprises checkpoint inhibitor therapy.

38. The method of claim 37, wherein the checkpoint inhibitor therapy comprises a PD-1 antibody.

39. The method of any one of claims 24-38, wherein the polypeptide or composition is administered systemically.

40. The method of claim 39, wherein the polypeptide or composition is administered by intravenous injection.

41. The method of any one of claims 24-40, wherein the administered dose of the cytotoxic agent is less than the minimum effective dose of the cytotoxic agent unlinked to collagen binding domain.

42. The method of any one of claims 24-41, wherein the administered dose of the cytotoxic agent is less than the minimum effective dose of the cytotoxic agent conjugated to an albumin polypetide or IgG Fc domain polypetide and unlinked to collagen binding domain.

43. The method of any one of claims 24-42, wherein the subject has been previously treated with a cytotoxic agent.

44. The method of claim 43, wherein the subject has been determined to be non-responsive to the previous treatment or wherein the wherein the subject experienced non-specific toxicity to the previous treatment.


SEQUENCE LISTING (21 sequences)

SEQ ID NO: 1 - protein, 205 aa, Homo sapiens
CSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSG

SEQ ID NO: 2 - protein, 193 aa, Unknown
  Description of Unknown collagen binding domain sequence
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRI

SEQ ID NO: 3 - protein, 791 aa, Artificial Sequence
  Description of Artificial Sequence Synthetic polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 4 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 5 - protein, 8 aa, Artificial Sequence
  Description of Artificial Sequence Synthetic peptide
GGGSGGGS

SEQ ID NO: 6 - protein, 40 aa, Artificial Sequence
  Description of Artificial Sequence Synthetic polypeptide
  MISC_FEATURE (1)..(40): This sequence may encompass 1-10 “Gly Gly Gly Ser” repeating units
  See specification as filed for detailed description of substitutions and preferred embodiments
GGGSGGGSGGGSGGGSGGGSGGGSGGGSGGGSGGGSGGGS

SEQ ID NO: 7 - protein, 609 aa, Unknown [sequence omitted]

SEQ ID NO: 8 - protein, 584 aa, Unknown
  Description of Unknown Albumin sequence
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 9 - protein, 608 aa, Unknown
  Description of Unknown Albumin sequence
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 10 - protein, 584 aa, Unknown
  Description of Unknown Albumin sequence
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALG

SEQ ID NO: 11 - protein, 10 aa, Bos sp.
LRELHLNNNC

SEQ ID NO: 12 - protein, 10 aa, Homo sapiens
LRELHLDNNC

SEQ ID NO: 13 - protein, 344 aa, Homo sapiens
CGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 14 - protein, 196 aa, Unknown
  Description of Unknown ECM sequence
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 15 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 228 aa, Homo sapiens
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 18 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 4 aa, Artificial Sequence
  Description of Artificial Sequence Synthetic peptide
GGGS

SEQ ID NO: 20 - protein, 4 aa, Artificial Sequence
  Description of Artificial Sequence Synthetic peptide
  See specification as filed for detailed description of substitutions and preferred embodiments
GGGS

SEQ ID NO: 21 - protein, 6 aa, Artificial Sequence
  Description of Artificial Sequence Synthetic 6xHis tag
HHHHHH